SEQUENCE LISTING (13 sequences)

SEQ ID NO: 1 - protein, 266 aa, HOMO SAPIENS
MRTEPLCGASPLLVPGDPYSVVVLLQGYAEPEGVGDAVRADGSVTLVLPQTRGPASSHRESPRGSGGAEAALEEAARGPILVDTGGPWAREALLGALAGQGVAPGDVTLVVGTHGHSDHIGNLGLFPGAALLVSHDFCLPGGRYLPHGLGEGQPLRLGPGLEVWATPGHGGQRDVSVVVAGTALGTVVVAGDVFERDGDEDSWQALSEDPAAQERSRKRVLVVADVVVPGHGPPFRVLREASQPETEGGGNSQQEPVVGDEEPALH

SEQ ID NO: 2 - DNA, 2031 bp, HOMO SAPIENS
agatactcgggacgctgaggcgggaggatcacatgagcccaggaggttgaggctgcagtgaggtatgatcgcgccactgcactcctgcctgggcaactgagcatgaaccctcctcaaagctaatggcgcttcaaaattctgcagtgcaaagaagttgctggggttgcggagcggagaaccgccaggaggctggagcccagccaggcagcctcctcgggcacacggtgaaggcgccggaactacagctacctccgcggcgccgcttcggccgccatatctgggtacacgggaaccgcagaggacaaactctcacccgcgttttcgatttctgcgctttcttcgagtgaggcaagacctagcagaggcccaaggtagagcgcggctaggattcgatggagggaaacataaaggatagcctttggagggtgacgggcaaggacgtacgtaccgcgaacggaatggggcgggggccgaggacgccgagggaggggcgggcccaagtgtgaagggagtctgggcgataccattttgggcaggggttgagggtgataccaacttaatgggaggcgggtggcagagaaccgaggcttaggggcagtggcggggccgagcgcgggtgggggatcgcggagggacaggacggtcgcccactgctccatttcctttctccccagcccgtccctccctgccaggagcagcctcatgcggaccgagccgctgtgcggggcatcccctctgctggtgcccggcgacccctactctgtggtggttctgctgcagggctacgcggagccagagggtgtgggcgatgccgtgcgcgccgacggctccgtgaccctggtcctaccccagacccggggcccggcctccagccaccgagagtccccgcgcgggagtggcggcgcagaggccgccctggaggaggcggcccgtggccccatcctggtggacaccgggggcccctgggctcgggaggcgctgctgggggcgctggcggggcagggcgtggccccgggagacgtgacgctagtggtggggacccacgggcactcggatcacatcgggaacttggggctgttcccaggcgcggctctgctggtctcgcacgacttctgccttcccggaggccgctacctgccccacgggctgggtgaggggcagcccctgcgcctgggcccggggctcgaggtgtgggccacgccgggccacgggggccagcgcgacgtgagcgtggtggtggccggcacggctctgggcaccgtggtggtggcgggagatgtgtttgagcgagatggggacgaggattcgtggcaggcactgagtgaagaccccgcagcccaggagcggagccggaagagggtcctggtcgttgccgacgtggtcgtacctggtcacgggcccccctttcgagtgttaagggaagcctcgcagcccgagacggagggtggagggaacagccagcaggagccggtggtcggagacgaggagcccgccctgcactaatcagcctcgagagggactgcactcttgtcagggaagccctaacagcgaagagctgctggagacagagtcagagcagtcaagggtgggagcttccagcccttccaggaggccagttttctagtgaagacagagtgcacctgacactgccatcacatcgtcagtatcactgcctgtctctgccaccaaactaagatcaaaggaggggctcagctccctgtgcattctccctgggcctcagtaaaatgggagaaggttcgtgggaggggcctccaaaataaaaatggaaactgcattgaaaatcatagtgccctagtgtaaatgtgaagcattgacaatgataatcgtgcaaacatggtaatctacaactacagcagaggcatactttggccaattgcccctttcttataggatggaggttgctgagagaaggtacagggcataaagatgaccccttagtcacctgacttatgtatttgttcatatttttttgtcacacaggctagagtgcagttgtgcagtcatagctca

SEQ ID NO: 3 - DNA, 21 bp, ARTIFICIAL SEQUENCE
  SYNTHETIC CONSTRUCT
ggaaacgaccgcaggtcgccg

SEQ ID NO: 4 - DNA, 20 bp, ARTIFICIAL SEQUENCE
  SYNTHETIC CONSTRUCT
gacagcgatagtttagtttc

SEQ ID NO: 5 - DNA, 18 bp, ARTIFICIAL SEQUENCE
  SYNTHETIC CONSTRUCT
ttgctggcgtccagcggc

SEQ ID NO: 6 - DNA, 20 bp, ARTIFICIAL SEQUENCE
  SYNTHETIC CONSTRUCT
gacagcgatagtttagtttc

SEQ ID NO: 7 - DNA, 18 bp, ARTIFICIAL SEQUENCE
  SYNTHETIC CONSTRUCT
tccctggcgtccagcggc

SEQ ID NO: 8 - DNA, 16 bp, ARTIFICIAL SEQUENCE
  SYNTHETIC CONSTRUCT
cgagcccctgcatcct

SEQ ID NO: 9 - DNA, 15 bp, ARTIFICIAL SEQUENCE
  SYNTHETIC CONSTRUCT
gccgcgcagcagaac

SEQ ID NO: 10 - DNA, 71 bp, MUS MUSCULUS
atgaacggtccagtgcgcaccgagcccctgcatggtgagatccctttgctggcgtccagcggctcctactc

SEQ ID NO: 11 - DNA, 71 bp, MUS MUSCULUS
tacttgccaggtcacgcgtggctcggggacgtaccactctagggaaacgaccgcaggtcgccgaggatcag

SEQ ID NO: 12 - protein, 43 aa, MUS MUSCULUS
MNGPVRTEPLHGEIPLLASSGSYSVVVLLRGYAEPQGAGDAVR

SEQ ID NO: 13 - protein, 42 aa, ARTIFICIAL SEQUENCE
  SYNTHETIC CONSTRUCT
MNGPVRTEPLHGEIPGVQRLLLRGGSAARLRGAAGSGRRGAC

(19) United States
(12) Patent Application Publication
(10) Pub. No.: US 2020/0046856 A1
(43) Pub. Date: Feb. 13, 2020

(54) METHODS FOR IDENTIFYING MBLAC1-DEPENDENT MOLECULAR NETWORKS

(71) Applicant: Florida Atlantic University Board of Trustees, Boca Raton, FL (US)

(72) Inventors: Randy D. Blakely, West Palm Beach, FL (US); Maureen K. Hahn, Jupiter, FL (US)

(21) Appl. No.: 16/057,013

(22) Filed: Aug. 7, 2018

(51) Int. Cl. A61K 49/00; A01K 67/027

(52) U.S. Cl. CPC A61K 49/0008; A01K 67/0276; A01K 2207/05; A01K 2217/075; A01K 2227/105

(57) ABSTRACT

Based on the discovery that MBLAC1 is a specific, high-affinity target for Ceftriaxone (Cef), MBLAC1 may be used for identifying molecules and molecular networks associated with MBLAC1's role in the actions of substances of abuse, e.g., molecules and networks of molecules modulated by Cef in an animal that expresses MBLAC1 and is administered a substance of abuse and not in an animal that does not express MBLAC although administered the same substance of abuse. Methods for identifying MBLAC1-dependent molecules and molecular networks include analyzing and comparing levels of molecules (nucleic acids, neurotransmitters, proteins, and metabolites) in MBLAC1 knock-out animals and wild-type MBLAC1 animals. These methods and MBLAC1 knock-out animals can also be used for identifying therapeutic drug targets for treatment of addiction to, or withdrawal from, a substance of abuse.

### FIELD OF THE INVENTION

The invention relates generally to the fields of pharmacology, medicine, neurology and psychiatry. In particular, the invention relates to methods for identifying molecules and networks of molecules in a mammal that are modulated by Cef in the presence of MBLAC1 but not in the absence of MBLAC1, and methods for identifying molecules and networks of molecules that are modulated by Cef in animals administered a substance of abuse in an MBLAC1-dependent manner.

### CROSS-REFERENCE TO A SEQUENCE LISTING

This application includes a “Sequence Listing” which is provided as an electronic document having the file name “6818-319_ST25.txt” (7,992 bytes, created Aug. 7, 2018), which is herein incorporated by reference in its entirety.

### BACKGROUND

Molecules bearing a four-membered β-lactam ring, typified by penicillin and the cephalosporins, are widely prescribed antibiotics (Van Boeckel et al., Lancet Infect Dis 14: 742-750, 2014). By interfering with cell wall synthesis, these agents halt bacterial cell division unless inactivated by β-lactamase proteins. Studies over the past decade have revealed that a number of these agents exhibit central nervous system (CNS) actions independent of their antimicrobial actions. One such study (Rothstein et al., Nature 433: 73-77, 2005), seeking opportunities to repurpose FDA-approved medications for the treatment of Amyotrophic Lateral Sclerosis (ALS), identified β-lactam antibiotics as candidates based on their ability to elevate expression of the glutamate (Glu) transporter, GLT-1. In particular, ceftriaxone (Cef), a CNS penetrant, cephalosporin-type, β-lactam antibiotic, showed high-potency in these studies. Subsequently, multiple groups have demonstrated Cef actions in a range of neurological and neurobehavioral disease models, including those for stroke, epilepsy, Parkinson's disease, and addiction, paralleled by normalization of pathological elevations in synaptic Glu levels. Cef has also been reported to elevate expression of the glial cystine/Glu exchanger (Xc−), thereby also normalizing extrasynaptic Glu levels. At present, the in vivo pharmacokinetics of existing inducers of GLT-1 such as Cef are poor (Rao et al., J Pers Nanomed 1(1):3-9, 2015), and the CNS target responsible for Cef action until recently was unknown.

### SUMMARY

In the experiments described below, Cef's brain target was identified, and understanding Cefs brain target now provides opportunities to develop new therapeutics for the many disorders attributed to Glu dysfunction, including addiction. Cef, a β-lactam antibiotic, has been reported to act independently of its antimicrobial actions to normalize perturbed CNS glutamate levels, principally by elevating expression of glial glutamate transporters. The experiments described below were driven by the hypothesis that identification of a specific, high-affinity target for Cef would significantly impact therapeutic development for multiple brain disorders, ranging from neurodegenerative disorders to addiction. Previously, a glial-expressed C. elegans gene, swip-10, was identified, that encodes a metallo-3-lactamase domain (MBD)-containing protein, and limits glutamate-dependent changes in dopamine neuron excitability. Bioinformatic analyses identified Mblac1 as the likely mammalian ortholog of swip-10. In the experiments described below, using cyanogen bromide immobilized Cef for affinity capture experiments and Backscattering Interferometry (BSI) to monitor MBLAC1 binding of unmodified Cef, evidence was obtained for specific, high affinity (K_{D}=2.2 μM) binding of Cef to MBLAC1. Moreover, it was found that specific immunodepletion of MBLAC1 from brain cytosolic extracts eliminated Cef binding activity. These studies support the hypothesis that MBLAC1 is the exclusive, high-affinity binding partner of Cef in the CNS, and show the path forward in the development of novel, MBLAC1-based therapeutics for the treatment of disorders where preclinical studies demonstrate Cef effectiveness, including models of substance abuse.

To gain insight into the functional role of MBLAC1 in vivo, CRISPR/Cas9 methods were used to disrupt N-terminal coding sequences of the mouse Mblac1 gene, resulting in a complete loss of protein expression in viable, homozygous knockout (KO) animals. Using serum from both wild-type (WT) and KO mice, global, untargeted metabolomic analyses were performed, resolving small molecules via hydrophilic interaction chromatography (HILIC) based ultra-performance liquid chromatography, coupled to mass spectrometry (UPLC-MS/MS). Unsupervised principal component analysis reliably segregated the metabolomes of MBLAC1 KO and WT mice, with 92 features subsequently nominated as significantly different by ANOVA, and for which tentative and putative metabolite assignments were made. Bioinformatic analyses of these molecules nominated validated pathways subserving bile acid biosynthesis and linoleate metabolism, networks known to be responsive to metabolic and oxidative stress. These results and similar experiments performed with brain extracts can be used to identify the substrate for MBLAC1 and how substrate hydrolysis supports the anti-addiction actions of Cef. These results demonstrate the use of MBLAC1 KO animals (e.g., rodents) for demonstrating specific pathways that can be activated or suppressed by loss of MBLAC1 and demonstrate how one can use MBLAC1 KO animals (e.g., rodents) to look for specific pathways that are dependent on MBLAC1 expression (e.g., pathways through which MBLAC1-targeted therapeutic drugs reduce the actions of substances of abuse).

Described herein are methods and kits for identifying and analyzing molecules and molecular networks associated with MBLAC1's role in the actions of substances of abuse, e.g., molecules and networks of molecules modulated by Cef in an animal that expresses MBLAC1 and is administered a substance of abuse and not in an animal that does not express MBLAC although administered the same substance of abuse. These methods can also be used for identifying therapeutic drug targets for treatment of addiction to, or withdrawal from, a substance of abuse. The data presented herein provide strong support for the assertion that the well-replicated actions of Cef in the CNS arise through MBLAC1 interactions. Based on these experimental results, MBLAC1 may be used for identifying MBLAC1-dependent molecules and molecular networks changed (modulated) by substances of abuse and/or Cef exposure that can inform on new targets for addiction medications. Analyzing gene/protein/metabolome/neurotransmitter/small molecule actions of specific substances of abuse using a MBLAC1 KO animal can identify pathways impacted by Cef and thus lead to targets for therapeutic drug screens.

Accordingly, described herein is a method of identifying molecular networks in an animal that are modulated by Cef in the presence of MBLAC1 but not in the absence of MBLAC1. The method includes the steps of: (a) providing a group of test MBLAC1 KO animals, a group of control MBLAC-1 KO animals, a group of test WT MBLAC1 animals and a group of control WT MBLAC1 animals, or cells from each group of animals; (b) administering a dose of Cef to the group of test MBLAC1 KO animals and to the group of test WT MBLAC1 animals, or to cells from the group of test MBLAC1 KO animals and to cells from the group of test WT MBLAC1 animals at least once under conditions known to reduce effects of a substance of abuse in an animal; (c) collecting biological samples from all the groups of animals and analyzing levels of nucleic acids, neurotransmitters, proteins, and metabolites in the biological samples or analyzing levels of nucleic acids, neurotransmitters, proteins, and metabolites in the cells from all the groups of animals, resulting in a plurality of test MBLAC1 KO molecular networks, a plurality of control MBLAC1 KO molecular networks, a plurality of test WT MBLAC1 molecular networks, and a plurality of control WT MBLAC1 molecular networks; (d) comparing the plurality of test WT MBLAC1 molecular networks to the plurality of control WT MBLAC1 molecular networks and identifying any molecular networks present in the test WT MBLAC1 animals but not in the control WT MBLAC1 animals as Cef-responsive molecular networks; (e) comparing the Cef-responsive molecular networks to the plurality of test MBLAC1 KO molecular networks and to the plurality of control MBLAC1 KO molecular networks and identifying any Cef-responsive molecular networks that are not present in the plurality of test MBLAC1 KO molecular networks or in the plurality of control MBLAC1 KO molecular networks, or that are overrepresented relative to the plurality of test MBLAC1 KO molecular networks, as molecular networks that are modulated by Cef in the presence of MBLAC1 but not in the absence of MBLAC1. In the method, the molecular networks that are modulated by Cef in the presence of MBLAC1 but not in the absence of MBLAC1 typically include a statistically significant number of molecules (nucleic acids, neurotransmitters, proteins, and/or metabolites) whose levels are modulated by Cef in the presence of MBLAC1 relative to molecules (nucleic acids, neurotransmitters, proteins, and/or metabolites) whose levels are not modulated by Cef in the absence of MBLAC1. The molecular networks that are modulated by Cef in the presence of MBLAC1 but not in the absence of MBLAC1 are typically associated with Cef's ability to act through MBLAC1 to reduce actions of at least one substance of abuse (e.g., cocaine), and are relevant to substance abuse and/or addiction. In a typical embodiment, steps (c)-(d) are performed using software (e.g., network software). The animals can be rodents (e.g., rats, mice), for example. The biological samples can be, for example, serum, cerebrospinal fluid, brain tissue, etc. In the method, analyzing levels of nucleic acids, neurotransmitters, proteins, and metabolites in step (c) can be performed, for example, by one or more of: RNA sequencing, microarray analysis, epigenome analysis, proteomic analysis and metabolomics analysis.

Also described herein is a method of analyzing MBLAC1-dependent levels of molecules (e.g., nucleic acids, neurotransmitters, proteins, and metabolites). The method includes analyzing levels of molecules in WT MBLAC1 animals or cells (isolated) therefrom and levels of molecules in MBLAC1 KO animals or cells (isolated) therefrom, comparing the levels of molecules in WT MBLAC1 animals or cells (isolated) therefrom to the levels of molecules in MBLAC1 KO animals or cells (isolated) therefrom and identifying any molecules whose levels of expression statistically significantly differ in the WT MBLAC1 animals or cells (isolated) therefrom relative to the MBLAC1 KO animals or cells (isolated) therefrom. Analyzing the levels of the molecules includes one or more of: RNA sequencing, microarray analysis, epigenome analysis, proteomic analysis and metabolomics analysis. The animals can be, for example, rodents (e.g., rats, mice). In the method, the identified molecules whose levels of expression statistically significantly differ in the WT MBLAC1 animals or cells therefrom relative to the MBLAC1 KO animals or cells therefrom form a molecular network.

Further described herein is a method of identifying molecules and/or molecular networks thereof modulated by a substance of abuse and Cef in an MBLAC1 dependent manner. The method includes the steps of: (a) providing a group of control WT MBLAC1 animals, a first test group of WT MBLAC1 animals, a second test group of WT MBLAC1 animals, a third test group of WT MBLAC1 animals, a group of control MBLAC1 KO animals, a first group of test MBLAC1 KO animals, a second test group of MBLAC1 KO animals and a third test group of MBLAC1 KO animals; (b) administering a substance of abuse to the first test group of WT MBLAC1 animals and collecting biological samples from the group of control WT MBLAC1 animals and from the first test group of WT MBLAC1 animals; (c) analyzing levels of molecules (nucleic acids, neurotransmitters, proteins, and metabolites) in the biological samples and determining differences in the levels of the molecules between the group of control WT MBLAC1 animals and the first test group of WT MBLAC1 animals; (d) administering a dose of Cef to the second test group of WT MBLAC1 animals and administering the same dose of Cef and the substance of abuse to the third test group of WT MBLAC1 animals, the dose of Cef being administered at least once under conditions known to reduce effects of the substance of abuse in an animal; (e) collecting biological samples from the second test group of WT MBLAC1 animals and the third test group of WT MBLAC1 animals after administration of the dose of Cef and the substance of abuse; (f) analyzing levels of molecules (nucleic acids, neurotransmitters, proteins, and metabolites) in the biological samples of step (e) and determining differences in the levels of the molecules between the second test group of WT MBLAC1 animals and the third test group of WT MBLAC1 animals; (g) comparing the differences in the levels of step (c) to the differences in the levels of step (f) to identify any molecules or one or more molecular networks thereof whose levels are modulated by Cef and by the substance of abuse; (h) administering a substance of abuse to the first test group of MBLAC1 KO animals and collecting biological samples from the group of control MBLAC1 KO animals and from the first test group of MBLAC1 KO animals; (i) analyzing levels of molecules (nucleic acids, neurotransmitters, proteins, and metabolites) in the biological samples of step (h) and determining differences in the levels of the molecules between the group of control MBLAC1 KO animals and the first test group of MBLAC1 KO animals; (j) administering a dose of Cef to the second test group of MBLAC1 KO animals and administering the same dose of Cef and the substance of abuse to the third test group of MBLAC1 KO animals, the dose of Cef being administered at least once under conditions known to reduce effects of the substance of abuse in an animal; (k) collecting biological samples from the second test group of MBLAC1 KO animals and the third test group of MBLAC1 KO animals after administration of the dose of Cef and the substance of abuse; (l) analyzing levels of molecules (nucleic acids, neurotransmitters, proteins, and metabolites) in the biological samples of step (k) and determining differences in the levels of the molecules between the second test group of MBLAC1 KO animals and the third test group of MBLAC1 KO animals; (m) comparing the differences in the levels of step (i) to the differences in the levels of step (l) to identify any molecules or one or more molecular networks thereof that modulate actions of the substance of abuse in any MBLAC1 KO animal that has been administered the substance of abuse; and (n) comparing the molecules or one or more molecular networks thereof whose levels are modulated by Cef and by the substance of abuse of step (g) to the molecules or one or more molecular networks thereof that modulate actions of the substance of abuse in any MBLAC1 KO animal of step (m) and identifying molecules or one or more molecular networks thereof whose levels are modulated by Cef and by the substance of abuse in an MBLAC1-dependent manner.

In the method, the substance of abuse can be any substance of abuse, e.g., cocaine, amphetamine, morphine, ethanol, methamphetamine, clorazepate, cathinones, bath salts, heroin, nicotine, alcohol, ketamine, MDMA, etc. Typically, the molecules or one or more molecular networks thereof whose levels are modulated by Cef and by the substance of abuse in an MBLAC1-dependent manner are relevant to substance abuse and/or addiction. In the method, steps (c), (f), (g), (i), (l), (m) and (n) are performed using network software. The animals can be, for example, rodents (e.g., rats, mice). The method can further include the step of identifying at least one (e.g., 1, 2, 5, 10, 50, 100, etc.) drug target from the molecules or one or more molecular networks thereof whose levels are modulated by Cef and by the substance of abuse in an MBLAC1-dependent manner. In some embodiments, the method further includes testing the at least one (e.g., 1, 2, 5, 10, 50, 100, etc.) drug target for an ability to attenuate or block the actions of the substance of abuse.

Additionally described herein is a method for identifying a potential therapeutic drug target for substance abuse treatment. The method includes identifying at least one molecule (nucleic acid, neurotransmitter, protein, or metabolite) that is modulated by a substance of abuse and Cef in an MBLAC1-dependent manner as identified in one of the methods described above as a potential drug target for treating abuse of the substance in a mammal. This method can further include screening candidate therapeutic drugs against the potential drug target.

Still further described herein is an MBLAC1 KO animal lacking any functional copies of the Mblac1 gene. In one embodiment, N-terminal coding sequences of the Mblac1 gene are disrupted by CRISPR/Cas9 resulting in a complete loss of MBLAC1 protein expression in the MBLAC1 knock-out animal. In an embodiment, mRNA transcription or mRNA translation of the Mblac1 gene has been significantly reduced or eliminated by chemical or genetic means. An MBLAC1 KO animal can be, for example, a rodent (e.g., rats, mice).

Yet further described herein is use of MBLAC1 KO animals (e.g., rodents such as mice and rats) to identify molecular pathways through which MBLAC1-targeted therapeutic drugs reduce the actions of substances of abuse in an animal (e.g., mammals, insects, fish).

As used herein, “protein” and “polypeptide” are used synonymously to mean any peptide-linked chain of amino acids, regardless of length or post-translational modification, e.g., glycosylation or phosphorylation.

By the terms “MBLAC1 protein” and “MBLAC1 polypeptide” is meant an expression product of a Mblac1 gene such as the native human MBLAC1 protein (UniprotKB Protein: A4D2B0), or a protein that shares at least 65% (but preferably 75, 80, 85, 90, 95, 96, 97, 98, or 99%) amino acid sequence identity with the foregoing. SEQ ID NO: 1 is the human MBLAC1 protein sequence.

As used herein, a “nucleic acid” or a “nucleic acid molecule” means a chain of two or more nucleotides such as RNA (ribonucleic acid) and DNA (deoxyribonucleic acid). By the term “gene” is meant a nucleic acid molecule that codes for a particular protein, or in certain cases, a functional or structural RNA molecule.

By the terms “Mblac1 gene,” “Mblac1 polynucleotide,” and “Mblac1 nucleic acid” is meant a native human MBLAC1-encoding nucleic acid sequence, e.g., the native human Mblac1 gene (RefSeq Accession: NC_000007.14), a nucleic acid having sequences from which a Mblac1 cDNA can be transcribed; and/or allelic variants and homologs of the foregoing. The terms encompass double-stranded DNA, single-stranded DNA, and RNA. SEQ ID NO: 2 is the human Mblac1 cDNA sequence. Within SEQ ID NO:2, base pairs 703-1503 are the coding sequence.

When referring to a nucleic acid molecule or polypeptide, the term “native” refers to a naturally-occurring (e.g., a WT) nucleic acid or polypeptide.

The terms “specifically binds to,” and “specific binding” refer to that binding which is characterized by either 1) having one member of a pair interact with the other species, but not other species at a comparable affinity (selectivity) and 2) having the detectable binding signal to the species eliminated when the species is absent, mutated to be non-functional or not expressed, or chemically-denatured, or when the species is bound with another molecule or compound already known to bind specifically to this species (competition).

The terms “percent identity” and “percent identical,” as known in the art, mean a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991). Preferred methods to determine identity are designed to give the best match between the sequences tested. Methods to determine identity and similarity are codified in publicly available computer programs. Sequence alignments and percent identity calculations may be performed using any suitable computer program.

The term “isolated” designates a biological material (small molecule, nucleic acid or protein) that has been removed from its original environment (the environment in which it is naturally present). For example, a protein present in its natural state in a plant or an animal is not isolated, however the same protein separated from the adjacent proteins in which it is naturally present, is considered “isolated”. The term “purified” means separated from many other entities (small molecules, proteins, nucleic acids, compounds), and does not require the material to be present in a form exhibiting absolute purity, exclusive of the presence of other entities. In some embodiments, a small molecule, compound, protein, nucleic acid or other entity is considered pure (purified) when it is removed from substantially all other entities.

By the terms “to modulate” and “modulates” is meant to increase or decrease. These terms can refer to increasing or decreasing an activity, level or function of a molecule (e.g., protein, peptide, nucleic acid, small molecule, metabolite), or effecting a change with respect to one or more biological or physiological mechanisms, effects, responses, functions, pathways or activities in which, for example, MBLAC1 and/or Cef are involved, such as a Cef-dependent signaling pathway or metabolic pathway.

When a molecule is referred to herein as “Cef-responsive”, that term means any molecule (nucleic acid (e.g., gene, RNA), poplypeptide, peptide, small molecule or metabolite) whose level or activity is modulated (i.e., increased or decreased) by Cef.

By the term “Cef-like” is meant any molecule with a four membered, beta-lactam ring or a substituted beta lactam ring.

As used herein, the phrases “network of molecules” and “molecular networks” mean a group or collection of two or more molecules related as molecules that bind to one another, as molecules that are precursor and product in a biochemical reaction, as regulators of each other or of common targets, or as molecules that are under the control of a common regulator. A molecular network can be a binding network whereby one or more molecules bind to a common target in the network, a functional network, supported by structural relationships (a molecular pathway) such that the action of one molecule can change the level or activity of a second, which can change the activity or level of a third. A molecular network can be a structural network. Speaking structurally, a molecule altered could be converted to another molecule whose activity or level changes and this alters a third molecule and so on. For example, a group of molecules in a structural network would be molecules in a biosynthetic pathway where X is converted to Y which is converted to Z. The changes in the latter molecules can have functional effects on other molecules (the first type of network). The network may be one related by co-expression such that when one molecule's expression changes the expression of the other molecule changes due to a response to a common regulator. Each of the proteomic, transcriptomic, genomic, and metabolomic approaches described herein identifies a collection of molecules or molecular differences occurring (associated) with the loss of MBLAC1. These molecules can be linked into a molecular network by software that looks for functional, structural or co-expression relationships in the literature.

By the term “drug target” is meant any molecule (e.g., nucleic acid, protein, peptide, small molecule, metabolite) that can be targeted by a therapeutic agent (therapeutic drug). In a typical embodiment, a drug target is a molecule that is causally involved in a disease process such as addiction or substance abuse, and a useful therapeutic drug is one that modulates the activity or amount of the molecule.

As used herein, “substance abuse” means the excessive use of a substance, especially alcohol or a drug, by an individual, e.g., excessive use of the drug or substance despite an understanding of the negative consequences in continued use (e.g. loss of job, loss of relationships, loss of health, loss of consciousness, loss of life).

As used herein, the terms “substance of abuse” and “drug of abuse” are used interchangeably and mean any substance or drug that is abused by an individual. Examples of substances of abuse include cocaine, amphetamine, morphine, ethanol, methamphetamine, clorazepate, cathinones, bath salts, heroin, nicotine, alcohol, ketamine, and MDMA.

The terms “agent” and “therapeutic agent” as used herein refer to a chemical entity or biological product, or combination of chemical entities or biological products, administered to a subject (a mammal such as a human) to treat a disease or condition (e.g., addiction). Examples of therapeutic agents include small molecules and biologics, which may be referred to herein as a “drug” or “therapeutic drug”.

The terms “patient,” “subject” and “individual” are used interchangeably herein, and mean a subject, typically a mammal, to be treated, diagnosed, and/or to obtain a biological sample from. Subjects include, but are not limited to, humans, non-human primates, horses, cows, sheep, pigs, rats, mice, dogs, and cats. A human in need of substance abuse or addiction treatment is an example of a subject.

The terms “sample,” “patient sample,” “biological sample,” and the like, encompass a variety of sample types obtained from a patient, individual, or subject and can be used in a therapeutic drug screening, diagnostic or monitoring assay. The patient sample may be obtained from a healthy subject, a diseased patient or a patient having associated symptoms of a particular disease or disorder (e.g., addiction to a substance(s) of abuse). Moreover, a sample obtained from a patient can be divided and only a portion may be used for therapeutic drug screening. Further, the sample, or a portion thereof, can be stored under conditions to maintain sample for later analysis. The definition specifically encompasses blood and other liquid samples of biological origin (including, but not limited to, cerebrospinal fluid, plasma, serum, peripheral blood, urine, saliva, stool and synovial fluid), solid tissue samples such as a biopsy specimen or tissue cultures or cells derived therefrom and the progeny thereof. In a specific embodiment, a sample includes a cerebrospinal fluid sample. In another embodiment, a serum sample is used. The definition also includes samples that have been manipulated in any way after their procurement, such as by centrifugation, filtration, precipitation, dialysis, chromatography, treatment with reagents, washing, or enriched for certain cell populations. The terms further encompass a clinical sample, and also include cells in culture, cell supernatants, tissue samples, organs, and the like. Samples may also comprise fresh-frozen and/or formalin-fixed, paraffin-embedded tissue blocks, such as blocks prepared from clinical or pathological biopsies, prepared for pathological analysis or study by immunohistochemistry.

As used herein, the terms “therapeutic treatment” and “therapy” are defined as the application or administration of a therapeutic agent or therapeutic agents to a patient who has a disease, a symptom of disease or a predisposition toward a disease, with the purpose to cure, heal, alleviate, relieve, alter, remedy, ameliorate, improve or affect the disease, the symptoms of disease, or the predisposition toward disease.

Although methods similar or equivalent to those described herein can be used in the practice or testing of the present invention, suitable methods are described below. All publications, patent applications, and patents mentioned herein are incorporated by reference in their entirety. In the case of conflict, the present specification, including definitions, will control. The particular embodiments discussed below are illustrative only and not intended to be limiting.

### BRIEF DESCRIPTION OF THE DRAWINGS

FIGS. 1A-1D show detection and expression of MBLAC1. 1A: Alignment of putative MBLAC1 orthologs across phylogeny. Except for nematodes, the MBD is the primary component of these proteins. Bars represent critical residues for metal binding and catalytic activity (“x” indicates any other amino acid). MBD positioning within each protein (aa=amino acids) was established with the SMART database online tool (acquisition number SM00849). 1B: Detection of mouse and human MBLAC1 from stably-transfected T-REx cells. Cells were induced (or not) with TET prior to generation, SDS-PAGE and Western blotting of cell lysates. Antibody #79 detects human and mouse MBLAC1 (top immunoblot IB), whereas antibody #80 can only detect the mouse isoform. β-Actin blots below verify that though loaded with equivalent protein, the non-TET cells do not express MBLAC1. IC: Subcellular fractionation of NIH 3T3 mouse fibroblast cell lysates reveal MBLAC1 expression is relegated to the cytosolic fractions, based on co-fractionation with GAPDH. 1D: Presence of MBLAC1 protein in various regions of the mouse brain. Bar graph shows normalized densitometries of bands over multiple experiments (n=6, one-way ANOVA shows no significant main effect of brain region (P>0.05).

FIGS. 2A and 2B show MBLAC1 binding to Cef-conjugated Sepharose beads. 2A: Schematic depicting Cef conjugation to CN—Br Sepharose beads via coupling to free amine group. 2B: Evidence of MBLAC1 binding to bead-conjugated Cef. Left panel shows immunoblot detection of total MBLAC1 input of T-REx cells±TET induction. Left panel shows immunoblot detection of MBLAC1 in SDS eluate from Cef-conjugated beads±preincubation of Cef (50 μM) with MBLAC1 containing extract. 2C: Quantitation of free Cef competition for binding of MBLAC1 to immobilized Cef (n=6, one-way ANOVA, Dunnett's multiple comparison test, **** (P<0.001)). Mean reduction in MBLAC1 binding in the presence of 50 μM free Cef is 52.4±9.3%.

FIGS. 3A-3F show results of a BSI analysis of MBLAC1 binding to unconjugated Cef. 3A: Lysates from TET induced T-REx cells expressing mouse MBLAC1 demonstrate concentration-dependent, heat-sensitive BSI binding signals that are absent from lysates prepared from cells lacking TET induction. (TET− vs TET+ at 5 μM and 50 μM, * (P<0.05) and **** (P<0.0001), respectively) 3B: Lysates from TET induced T-REx cells expressing human MBLAC1 demonstrate concentration-dependent, heat-sensitive BSI binding signals that are absent from lysates prepared from cells lacking TET induction. (TET− vs TET+ at 5 μM and 50 μM, *** (P<0.001) and **** (P<0.0001), respectively). 3C: Estimation of binding stoichiometry and Cef binding affinity to lysate from mouse MBLAC1 expressing cells using BSI. Single site binding equation fit to BSI binding data (r²=0.96) yields a K_{D} for binding of 2.2 μM±0.56. 3D: In contrast to Cef binding, multiple concentrations of CephC result in a non-significant reduction in BSI signal using lysates of mouse MBLAC1 expressing cells (TET− vs TET+ at 50 μM Cef **** (P<0.0001), TET− vs TET+ at 5 μM or 50 μM CephC, P>0.05). 3E: Immunoblot of mouse frontal cortex extracts subjected to immunodepletion with MBLAC1 Ab #80 compared to IgG control immunodepleted extracts. 3F: Lysate of mouse frontal cortex shows binding signal with Cef that was abolished when MBLAC1 was immunodepleted using MBLAC1 antibody #80, or when the samples were heat inactivated prior to BSI analysis (Lysate vs depleted lysate at 5 μM and 50 μM, ** (P<0.01) and **** (P<0.0001), respectively). All experiments were analyzed using a two-way ANOVA and Tukey multiple comparisons tests.

FIGS. 4A-4D show CRISPR/Cas9 generation of the MBLAC1 KO mouse. 4A: Gene diagram depicts the target sequence used to direct DNA cut sites in the Mblac1 genomic sequence. The protospacer adjacent motif (PAM) and protospacer sequences are highlighted and 5 bp deletion and 14 bp deletion of the KO are underlined. The top nucleic acid sequence is SEQ ID NO: 10 and the bottom nucleic acid sequence is SEQ ID NO: 11. 4B: Beginning of the protein sequences for WT and the 5 bp MBLAC1 KO, highlighting the frameshift/missense amino acid sequence and early truncation of the 5 bp MBLAC1 KO line generated and used in the present study. The top protein sequence is SEQ ID NO: 12 and the bottom protein sequence is SEQ ID NO: 13. 4C: MBLAC1 immunoblot of protein lysates prepared from WT and KO brain (cortical tissue) and liver tissue. MBLAC1 KO mouse tissue lacks the specific 27 kDa MBLAC1 band.

FIG. 5 is an illustration of the workflow for the global, untargeted MBLAC1 KO serum metabolomic discovery and validation studies. The workflow begins with serum sample preparation from age- and sex-matched controls for the discovery set, and serum sample preparation from littermate age- and sex-matched controls for the validation set. This diagram illustrates the steps required for the discovery-based analysis of a multidimensional dataset across several analysis platforms to curate tentative and putative feature annotations and prioritize metabolic pathways altered by loss of MBLAC1. Additionally, this illustration describes the validation analysis to identify replicable metabolic pathways sensitive to MBLAC1 loss.

FIG. 6 is a graph showing the effect of repeated Cef on cocaine-induced locomotion. Mice were injected IP once daily for 10 days with 200 mg/kg CEF or vehicle. Twenty-four hours following the last Cef injection, mice were injected with 10 mg/kg cocaine or vehicle followed immediately by testing in open field activity chambers for 60 minutes. Distance travelled (cm) in the first 30 min following cocaine injection was calculated and data are expressed as the mean+/−SEM for each group. *p<0.05, Student's t-test. CEF, ceftriaxone; COC, cocaine, HOM, homozygote; WT, wild-type.

FIGS. 7A and 7B are a pair of graphs showing the effect of repeated cocaine on open field activity. HOM mice demonstrate an enhanced response to the effects of cocaine, and an increased development of cocaine-induced sensitization. Mice were injected with 10 mg/kg cocaine for five consecutive days, and once two weeks later, followed immediately after each injection by recording of activity in open field activity chambers for 60 minutes. Ambulatory distance travelled (cm) in the total 60 min period following cocaine injection was calculated and data are expressed as the mean+/−SEM for each group. *p<0.05, ***p<0.001, ****p<0.0001, Tukey's multiple comparisons test. HOM, homozygote; WT, wild-type.

### DETAILED DESCRIPTION

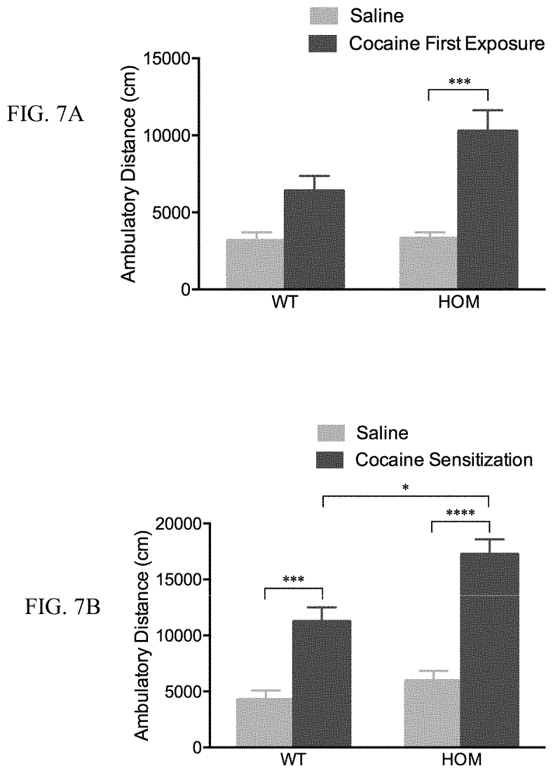

The β-lactam compound Cef, and several other structural analogs, have been shown to reduce the ability of substances of abuse (e.g., cocaine, amphetamine, morphine, ethanol, methamphetamine, clorazepate, cathinones, bath salts, heroin, nicotine, alcohol, ketamine, and MDMA) to impact behavior, either upon initial administration, upon chronic administration, or following withdrawal. This action arises through complex mechanisms that appear to be unrelated to the agent's actions as an antibiotic. Rather, studies indicate an indirect ability of Cef to modulate extracellular glutamate (Glu) levels via induction of one or more cell surface Glu transporters, effects that are ultimately believed to influence signaling by dopamine (DA) in brain reward centers. Described herein is the discovery that Cef binds to the protein MBLAC1. Previously it was shown that mutation of the putative C. elegans ortholog of MBLAC1, SWIP-10, leads in worms to changes in Glu-dependent activation of DA neurons. To investigate the effects of loss of MBLAC1 in mammals and the requirement for MBLAC1 in the actions of drugs of abuse, MBLAC1 KO mice were developed, and in these MBLAC1 KO, a stronger locomotor response to cocaine compared to WT littermates was observed (FIG. 7A, 7B), as well as a stronger response after withdrawal and readministration (sensitization) revealing a likely impact on psychostimulant-induced brain plasticities. Additionally, cocaine (10 mg/kg) injections into WT mice pretreated for 10 days with saline showed pronounced locomotor activation, with equivalent effects seen with cocaine injections give to MBLAC1 KO mice pretreated with saline. When cocaine injections to WT animals were preceded by a 10 day Cef treatment (200 mg/kg; once a day, i.p.), the locomotor activating effects of cocaine were lost. In contrast, Cef displayed no ability to attenuate cocaine actions in the MBLAC1 KO mice. Together, these findings indicate that loss of MBLAC1 expression impacts brain mechanisms constraining actions of cocaine and that MBLAC1 expression is required for the ability of Cef to exert its inhibitory actions on cocaine psychomotor activation. These data are the first evidence that MBLAC1-dependent pathways participate in, and can be pharmacologically targeted to modulate the actions of drugs of abuse. These data implicating a requirement for MBLAC1 to achieve suppression of cocaine action, described in more detail in the Examples below, support the utility of using a MBLAC1 KO animal to identify addiction-associated gene and protein networks (networks of molecules), and thereby new targets for anti-addiction development. The methods described herein are useful for identifying a biomarker(s) that indicates substance abuse or addiction risk, identifying biomarkers for a successful therapeutic drug response that can predict efficacy during therapeutic drug development for a potential addiction treatment, and to develop a drug target for a new therapeutic drug for addiction. Based on the drug target, therapeutic molecules can be developed through de novo molecular drug design, or from, for example, compound libraries and tested in vitro. A drug target identified by the methods and MBLAC1 KO animals described herein can be used to screen for therapeutic drug candidates and to identify lead compounds in therapeutic drug development programs.

Generally, the individual elements (molecules) of a molecular network, or multiple molecules in the molecular network together, or their qualitative or quantitative relationships, can be used as 1) a biomarker of substance of abuse action—an indication that the substance of abuse is acting in an MBLAC1-dependent manner and likely to be treatable by a chemical identified to bind or modulate MBLAC1 (analogous to Cef) and 2) a biomarker of treatment response—an indication that in a subject addicted to a substance of abuse, a network of molecules is changing in levels or relationships as it would were Cef being used as the treatment. So once a molecule or molecular network is identified, then levels of that molecule(s) or their relationships are quantified in a biological sample (e.g., serum, urine or tissue extracts) from 1) a person that self-administers a substance of abuse or 2) a person undergoing treatment with a novel chemical (medication) identified as binding to MBLAC1, affecting the expression of MBLAC1, or evoking changes in in vitro or in vivo physiology in a manner similar to that arising from Cef treatment.

### Methods of Analyzing Molecules and Molecular Networks that are Modulated in an MBLAC1-Dependent Manner

The methods described herein include identifying molecules that are over or under represented (expressed) in WT MBLAC1 animals relative to MBLAC1 KO animals. These methods also include identifying molecular networks containing such molecules. In some embodiments, an identified molecular network that is found in WT MBLAC1 animals is not present in MBLAC KO animals. In typical methods, once a molecule or molecular network is identified, it can lead to a screen for candidate therapeutic drugs that are able to alter (e.g., attenuate or block) the acute or chronic actions of a substance of abuse. Acute and chronic actions of substances of abuse in animals are well known, and depending on the particular substance of abuse, can include altered locomotor activity, changes in expression and/or activity of molecules, changes in behavior associated with drug (substance of abuse) liking (e.g., lever pressing to obtain drug) or drug seeking (moving to an area where drug is provided), continuing to behave in a way that drug (substance of abuse) can be delivered even though the drug is no longer provided (e.g. continuing to lever press when no drug is available), and changes in body temperature, respiration, pupillary dilation, sweating, or GI function. In some embodiments, one or molecules within an identified molecular network are targets for therapeutic drug development. Methods of screening for and testing candidate therapeutic drugs are well known in the art and are described below. Since MBLAC1 is required for the action of Cef with regard to substance abuse, identifying molecules/networks of molecules dependent on MBLAC1 expression can be used to identify useful molecules, pathways and molecular networks that may underlie drug abuse action, serve as markers of successful treatment, and support identification of an anti-addictive treatment.

A typical method of identifying molecular networks in an animal that are modulated by Cef in the presence of MBLAC1 but not in the absence of MBLAC1 includes providing a group of test MBLAC-1KO animals, a group of control MBLAC-1 KO animals, a group of test wild-type WT MBLAC1 animals and a group of control WT MBLAC1 animals, or cells from each group of animals. A dose of Cef is administered to the group of test MBLAC1 KO animals and to the group of test WT MBLAC1 animals, or to cells from the group of test MBLAC1 KO animals and to cells from the group of test WT MBLAC1 animals at least once under conditions known to reduce effects of a substance of abuse in an animal. Biological samples are collected from all the groups of animals and levels of nucleic acids, neurotransmitters, proteins, and metabolites in the biological samples are analyzed, or levels of nucleic acids, neurotransmitters, proteins, and metabolites in the cells from all the groups of animals are analyzed. These analyses result in a plurality of test MBLAC1 KO molecular networks, a plurality of control MBLAC1 KO molecular networks, a plurality of test WT MBLAC1 molecular networks, and a plurality of control WT MBLAC1 molecular networks. The plurality of test WT MBLAC1 molecular networks are compared to the plurality of control WT MBLAC1 molecular networks and any molecular networks present in the test WT MBLAC1 animals but not in the control WT MBLAC1 animals are identified as Cef-responsive molecular networks. The Cef-responsive molecular networks are compared to the plurality of test MBLAC1 KO molecular networks and to the plurality of control MBLAC1 KO molecular networks and any Cef-responsive molecular networks that are not present in the plurality of test MBLAC1 KO molecular networks or in the plurality of control MBLAC1 KO molecular networks, or that are overrepresented relative to the plurality of test MBLAC1 KO molecular networks, are identified as molecular networks that are modulated by Cef in the presence of MBLAC1 but not in the absence of MBLAC1. One or more of these steps can be performed using software, e.g., network software. The animals can be any type of animal, e.g., non-human primates, horses, cows, sheep, pigs, rats, mice, dogs, fish, worms, and cats. In the method, typically the molecular networks that are modulated by Cef in the presence of MBLAC1 but not in the absence of MBLAC1 include a statistically significant number of nucleic acids, neurotransmitters, proteins, and/or metabolites (molecules) whose levels are modulated by Cef in the presence of MBLAC1 relative to nucleic acids, neurotransmitters, proteins, and/or metabolites (molecules) whose levels are not modulated by Cef in the absence of MBLAC1. The biological samples can be any suitable biological sample, e.g., serum, cerebrospinal fluid and brain tissue. In the method, analyzing the levels of nucleic acids, neurotransmitters, proteins, and metabolites (molecules) can be done by any suitable methods. Examples of well-known suitable methods include RNA sequencing, microarray analysis, epigenome analysis, proteomic analysis and metabolomics analysis. Methods of analyzing human metabolomics are known and described in, for example, Ramautar et al., Current Opinion in Chemical Biology 17(5):841-846, 2013; Mangalam et al., J Clin Cell Immunol. Vol. 4, Jun. 30, 2013; L. Puchades-Carrasco & A. Pineda-Lucena Curr Top Med Chem 17(24):2740-2751, 2017; G. W. Caldwell & G. C. Leo Curr Top Med Chem 17(24)2716-2739, 2017; Rattray et al. Curr Pharmacol Rep 3(3):114-125, 2017; and U.S. Pat. Nos. 7,005,255 and 9,977,034, all of which are incorporated herein by reference in their entireties.

In some embodiments, the molecules or molecular networks that are modulated by Cef in the presence of MBLAC1 but not in the absence of MBLAC1 are associated with Cef's ability to act through MBLAC1 to reduce actions of at least one substance of abuse, and are relevant to at least one of substance abuse and addiction.

As used herein, the phrase “under conditions known to reduce effects of a substance of abuse in an animal” means under conditions known to those of skill in the art that demonstrate Cef's actions to attenuate the actions of a substance off abuse. For example, the assay described in Example 3 is a cocaine-induced locomotion assay in which Cef is administered at a dose and time points known to block the effects of cocaine on mice (block the animal's response to cocaine)—i.e., under conditions known to reduce (attenuate, block) the effects of cocaine in a rodent. The methods were performed essentially as described in Tallarida et al. Neurosci Lett vol. 556, p. 155-159, 2013, which is incorporated by reference herein in its entirety. Numerous studies show the efficacy of Cef in various animal models examining the actions of drugs of abuse; these studies describe the conditions under which Cef reduces (attenuates) the effects of a substance of abuse in animals. These studies test one or more of: 1) acute sensitivity to the drug, 2) development of tolerance, 3) development of sensitization, 4) development of dependence, 5) reinstatement to drug after withdrawal, and 6) pathological changes in brain due to drug action. Examples of these studies include: Bell et al., Neuropharmacology. 2017 Aug. 1; 122:201-243; Rawls et al., Eur J Pharmacol. 2008 Apr. 28; 584(2-3):278-84; Wang et al., Yao Xue Xue Bao. 2008 November; 43(11):1094-8; Sari et al., J Neurosci. 2009 Jul. 22; 29(29):9239-43; Knackstedt et al., Biol Psychiatry. 2010 Jan. 1; 67(1):81-4; Rawls et al., Behav Pharmacol. 2010 March; 21(2):161-4; Ward et al., Behav Pharmacol. 2011 August; 22(4):370-3, etc. Based on these and many other publications, one of skill in the art would know under what conditions Cef reduces the effects of one or more substances of abuse.

The selection of an appropriate software program, e.g., a network software program, is within the ordinary skill of the art. It should be noted that the comparison of molecular networks can be done both quantitatively and qualitatively. In one embodiment, the invention pertains to the creation of addiction-associated molecular networks databases containing information regarding the genome, transcriptome, proteome, and metabolome of cells, cellular compartments, and organelles in healthy, diseased, and altered states. By the term “network software” is meant any software suitable for analyzing the possibility that two or molecules are related by structure, by function, by regulating a common target, by being the target of a common regulator, or having their expression modulated by a common factor. Networks for structurally, functionally or coexpression networks are tabulated in, for example, one or more of: KEGG, WebGestalt, STRING, GeneMANIA, OMICTools, NetworkAnalyst, Ingenuity, and a number of others to use for statistical evaluation. Methods of using network software are known in the art and described in, for example, Simmler et al., Br J. Pharmacol. 2017 174(16):2716-2738; and Muller et al., J. Neuropsychopharmacology 2017 42(2):427-436.

Examples of drugs or substances of abuse include cocaine, amphetamine, morphine, ethanol, methamphetamine, clorazepate, cathinones, bath salts, heroin, nicotine, alcohol, ketamine, and MDMA. In addition to these specific examples, a drug or substance of abuse is any drug or substance abused by an individual and in some cases, shown to be sensitive to Cef administration in rodents.

A typical method of analyzing MBLAC1-dependent levels of molecules includes analyzing levels of molecules in WT MBLAC1 animals or cells therefrom and levels of molecules in MBLAC1 KO animals or cells therefrom, comparing the levels of molecules in WT MBLAC1 animals or cells therefrom to the levels of molecules in MBLAC1 KO animals or cells therefrom and identifying any molecules whose levels of expression statistically significantly differ in the WT MBLAC1 animals or cells therefrom relative to the MBLAC1 KO animals or cells therefrom. In the method, a molecule is typically a nucleic acid, neurotransmitter, protein, or a metabolite. As with all methods described herein, the animals can be any type of animal, and the levels of molecules can be analyzed by any suitable methods, e.g., RNA sequencing, microarray analysis, epigenome analysis, proteomic analysis and metabolomics analysis. In some embodiments, the identified molecules whose levels of expression statistically significantly differ in the WT MBLAC1 animals or cells therefrom relative to the MBLAC1 KO animals or cells therefrom form a molecular network.

In some embodiments of methods of analyzing MBLAC1-dependent levels of molecules and of identifying molecular networks in an animal that are modulated by Cef in the presence of MBLAC1 but not in the absence of MBLAC1, cells isolated from MBLAC1 KO and WT MBLAC1 animals can be used (analyzed), e.g., primary cultures from WT MBLAC1 and MBLAC1 KO animals. Any appropriate types of cells (e.g. cultured astrocytes, embryonic fibroblasts (MEFs)) can be isolated from the animals. In these embodiments, once the cells have been treated with a substance of abuse, either acutely (e.g. 1 hr) or chronically (e.g., 1-7 days), they are analyzed substantially the same way that biological samples (e.g., tissue samples) from the animals are.

In the methods described herein, a molecular network can have several significantly changed (modulated) molecules (e.g., nucleic acids, proteins, metabolites, neurotransmitters) linked together with other molecules that are not themselves changed (modulated). The statistical question that is asked is whether the molecular network has more changed (modulated) molecules in it, given its size, than is expected by chance. For example, a molecular network might be known (or set) to contain of 100 interconnected molecules and with a 0.05 type I error used, one would expect 5 of these molecules to have demonstrated significance (up or down) by chance. If one identifies 10 molecules within this molecular network, this can be evaluated statistically to determine whether this is enough to determine whether it is statistically likely that this molecular network has more molecules with significant differences than expected by chance. If so, this will implicate or identify this molecular network as one where new targets may be identified for therapeutic drug development, including the molecules that were found to contribute within the molecular network. Whether the change from 5 to 10 in the example above is significant will depend on how many other molecular networks are being tested statistically in parallel. The more molecular networks being tested, the more likely it is that any one molecular network will show a significant number of molecules that are overrepresented (i.e., molecules whose levels are increased or decreased relative to non-substance of abuse-treated animals). One common (Bonferroni) correction is to multiply the P value from the initial assessment of significant overrepresentation within the molecular network by the number of tests being done. Thus, for one molecular network being assessed at a 0.05 level of significance, if one found by testing that the P value for the network with 10 differences versus chance expectations (5) is 0.01 and one is testing 50 molecular networks, the adjusted p value is 0.01×50=0.5. This level of significance does not exceed one's accepted level of type 1 error and so you one is not allowed to claim that the fact that one molecular network changes is, in toto, an indication of a non-random effect.

In some embodiments of identifying a molecular network(s), an identified network is one of biomarkers including DNA modifications (epigenome), RNA level changes (transcriptome), protein level changes (proteome), protein modifications (phosphorylation, acetylation, sulfation etc), and small molecule changes (metabolome) that are impacted by the presence or absence of MBLAC1. In the experiments described in Example 2 below, it was shown that MBLAC1 is required for the ability of Cef to block the actions of a substance of abuse (cocaine). This suggests that MBLAC1 normally supports the production of a molecule or network of molecules that Cef can alter (modulated) in abundance or structure to influence the actions of drugs of abuse. There are networks of serum molecules identified using the MBLAC1 KO and liquid chromatography/mass spectrometry (LC-MS/MS) showing feasibility and approach. In this approach, no substance of abuse is administered; networks of molecules in WT MBLAC1 and MBLAC1 KO mice are compared. In Example 2, serum was analyzed, but the same assessments can be made using, for example, urine, tissue or fluid from the brain or specific brain regions associated with substance abuse (e.g. nucleus accumbens, frontal cortex). With this information, one can evaluate these molecules or networks of molecules for potential targets of Cef due to its interaction with MBLAC1. In other embodiments, the substance of abuse (e.g. cocaine) is administered to either a WT MBLAC1 or MBLAC1 KO animal and Cef is administered before or after this substance (e.g. cocaine) to reduce onset or withdrawal/relapse respectively. In this case, analysis of the serum, brain fluid or brain tissue can be profiled (analyzed) by the proteomic, transcriptomic, genomic, and metabolomic approaches noted above to identify molecules or networks of molecules that underlie the ability of MBLAC1 to respond to Cef in the context of drug administration. See Example 3 describing an experiment involving rodents and onset with cocaine and Cef administration before cocaine was administered.

In the methods described herein, networks of molecules that are changed by abused drug administration and then modulated (i.e., levels of more molecules in the network than expected by chance are increased or decreased) with Cef treatment of WT MBLAC1 animals but not MBLAC1 KO animals would be prioritized as informative of those networks of molecules likely to be ones connected to (associated with) Cef's actions on the drug of abuse. By administering a substance of abuse and Cef to these animals, one can examine changes that occur when comparing the substance of abuse alone to the substance of abuse+Cef. i.e., what change is predicting Cef's ability to reduce the action of a substance of abuse in an MBLAC1 dependent manner. In one example of a method, molecules/networks of molecules are identified by comparing substance of abuse administration vs saline administration in WT MBLAC1 animals. One then identifies subsets of these networks that are altered when this comparison is done using MBLAC1 KO mice. This reveals molecules/networks of molecules that are directly associated with the substance of abuse that are sensitive to MBLAC1 expression. In an additional comparison, one can generate networks of molecules by administering substance of abuse+Cef in WT MBLAC1 animals and comparing these to molecule/molecular networks MBLAC1 KO animals having received the substance abuse+Cef. By comparing these two analyses, one can identify the different networks of molecules that relate to Cef action on an animal's response to a substance of abuse in an MBLAC1-dependent manner. For example, a method of identifying molecules or networks of molecules modulated by a substance of abuse and Cef in an MBLAC1-dependent manner can include the following groups of animals:

1) WT MBLAC1 saline

2) WT MBLAC1 drug of abuse

3) WT MBLAC1 saline and Cef

4) WT MBLAC1 drug of abuse and Cef

5) MBLAC1 KO saline

6) MBLAC1KO drug of abuse

7) MBLAC1KO saline and Cef

8) MBLAC1KO drug of abuse and Cef

Each of these 8 groups of animals would have “Omics” (metabolomics, proteomics, genomics, transcriptomics) analyses applied to them, and several comparisons of the molecules/molecular networks identified can be performed. In one example of such a comparison analysis, molecules and their molecular networks that change (are modulated) when Cef is administered to WT animals but not when Cef is administered to MBLAC1 KO animals, then these serve as targets for drug development, and then these targets lead to candidate therapeutic drugs and their improvements are used to suppress actions of substances of abuse. The comparison analysis determines differences between groups 1) and 3) and compares these results to the differences found when groups 5) vs 7) are compared. In another example of a comparison analysis, molecules where the substance of abuse is found to act differently between WT animals and KO animals in the absence of Cef are identified. This determines the difference between groups 1) and 2) and compares this to groups 5) and 6). This indicates that the molecule/molecular network is dependent on MBLAC1 in a normal response to a substance of abuse. This comparison analysis allows for identifying actions that MBLAC1 does that Cef acting on MBLAC1 might not. In yet another example of a comparison analysis, molecules and their molecular networks that change when Cef and a substance of abuse are present in WT MBLAC1 animals but not in MBLAC1 KO animals. For example, one determines first the difference between groups 1) and 2) and secondly the differences between groups 3) and 4). These two sets of differences are then compared to give the molecule/molecular networks that are impacted by the substance of abuse vs saline and that also are impacted by Cef vs no substance of abuse. Then one repeats this analysis using the MBLAC1 KO animals. One first evaluates groups 5) and 6), obtains that difference of molecule/molecular networks and then evaluate groups 7) and 8). One compares these two sets of differences to give the molecules/molecular networks that impact the actions of a substance of abuse vs saline in an MBLAC1 KO animal. One takes the molecule/molecular networks that are impacted by the substance of abuse vs saline and that also are impacted by Cef vs no substance of abuse and compares them to the molecule/molecular networks that impact the actions of the substance of abuse vs saline in an MBLAC KO animal. This generates (results in) the molecules/molecular networks that are sensitive to the substance of abuse vs saline, that are impacted by Cef differently than saline, and that require MBLAC1 to yield those effects.

In one embodiment of such a method of identifying molecules and/or molecular networks thereof modulated by a substance of abuse and Cef in an MBLAC1 dependent manner, the method includes the following steps:

(a) providing a group of control WT MBLAC1 animals, a first test group of WT MBLAC1 animals, a second test group of WT MBLAC1 animals, a third test group of WT MBLAC1 animals, a group of control MBLAC1 KO animals, a first group of test MBLAC1 KO animals, a second test group of MBLAC1 KO animals and a third test group of MBLAC1 KO animals;

(b) administering a substance of abuse to the first test group of WT MBLAC1 animals and collecting biological samples from the group of control WT MBLAC1 animals and from the first test group of WT MBLAC1 animals;

(c) analyzing levels of molecules selected from the group consisting of: nucleic acids, neurotransmitters, proteins, and metabolites in the biological samples and determining differences in the levels of the molecules between the group of control WT MBLAC1 animals and the first test group of WT MBLAC1 animals;

(d) administering a dose of Cef to the second test group of WT MBLAC1 animals and administering the same dose of Cef and the substance of abuse to the third test group of WT MBLAC1 animals, wherein the dose of Cef is administered at least once under conditions known to reduce effects of the substance of abuse in an animal;

(e) collecting biological samples from the second test group of WT MBLAC1 animals and the third test group of WT MBLAC1 animals after administration of the dose of Cef and the substance of abuse;

(f) analyzing levels of molecules selected from the group consisting of: nucleic acids, neurotransmitters, proteins, and metabolites in the biological samples of step (e) and determining differences in the levels of the molecules between the second test group of WT MBLAC1 animals and the third test group of WT MBLAC1 animals;

(g) comparing the differences in the levels of step (c) to the differences in the levels of step (f) to identify any molecules or one or more molecular networks thereof whose levels are modulated by Cef and by the substance of abuse;

(h) administering a substance of abuse to the first test group of MBLAC1 KO animals and collecting biological samples from the group of control MBLAC1 KO animals and from the first test group of MBLAC1 KO animals;

(i) analyzing levels of molecules selected from the group consisting of: nucleic acids, neurotransmitters, proteins, and metabolites in the biological samples of step (h) and determining differences in the levels of the molecules between the group of control MBLAC1 KO animals and the first test group of MBLAC1 KO animals;

(j) administering a dose of Cef to the second test group of MBLAC1 KO animals and administering the same dose of Cef and the substance of abuse to the third test group of MBLAC1 KO animals, wherein the dose of Cef is administered at least once under conditions known to reduce effects of the substance of abuse in an animal;

(k) collecting biological samples from the second test group of MBLAC1 KO animals and the third test group of MBLAC1 KO animals after administration of the dose of Cef and the substance of abuse;

(l) analyzing levels of molecules selected from the group consisting of: nucleic acids, neurotransmitters, proteins, and metabolites in the biological samples of step (k) and determining differences in the levels of the molecules between the second test group of MBLAC1 KO animals and the third test group of MBLAC1 KO animals;

(m) comparing the differences in the levels of step (i) to the differences in the levels of step (l) to identify any molecules or one or more molecular networks thereof that modulate actions of the substance of abuse in any MBLAC1 KO animal that has been administered the substance of abuse; and

(n) comparing the molecules or one or more molecular networks thereof whose levels are modulated by Cef and by the substance of abuse of step (g) to the molecules or one or more molecular networks thereof that modulate actions of the substance of abuse in any MBLAC1 KO animal of step (m) and identifying molecules or one or more molecular networks thereof whose levels are modulated by Cef and by the substance of abuse in an MBLAC1-dependent manner. In the method, the action of the substance of abuse may require the substance of abuse be given acutely (e.g., 1 hr) as for locomotion effects, to be given chronically for example 1-7 days (as for a response typical of addiction) or given chronically and then removed (measures of withdrawal and relapse).

Genes, RNAs, proteins, and metabolites identified as dependent on the presence of MBLAC1 in a molecular network (so statistically different comparing WT MBLAC1 and MBLAC KO) are all potential targets (drug targets) for therapeutic drug targeting. In a typical method of identifying a potential therapeutic drug target for substance abuse treatment, the method includes identifying at least one nucleic acid, neurotransmitter, protein, or metabolite that is modulated by a substance of abuse and Cef in an MBLAC1-dependent manner as identified in one or more of the methods described above as a potential drug target for treating abuse of the substance in a mammal. In some embodiments, the method further includes screening candidate therapeutic drugs against the potential drug target. The method can further include testing an identified drug target for an ability to attenuate or block the actions of a particular substance of abuse.

In one embodiment of a method of identifying a MBLAC-1-dependent network modulated by at least one substance of abuse, individual levels of RNAs, genes, proteins, and metabolites in WT MBLAC1 animals and MBLAC1 KO animals are quantified by standard assays (e.g., RNA sequencing or microarray, proteomic analysis, metabolomic analysis, etc). Once these individual levels are obtained, network software is used to see if there are specific molecular networks, consisting of two or more (e.g., 2, 3, 4, 5, 10, 15, 20, 25, 50, 100, etc.) of these RNAs, proteins, genes or metabolites, that are overrepresented in Cef-administered WT MBLAC1 animals vs Cef-administered MBLAC1 KO animals, using groups of each type of animal such that some groups receive the substance of abuse and some do not (e.g., one or more control groups). Generally, these methods are used to identify a molecular network(s) that is reduced in significance (e.g., a molecular network in which levels of certain genes, RNAs, proteins, neurotransmitters or metabolites are reduced) by Cef in WT MBLAC1 animals but not in MBLAC1 KO animals. Using these animals, one can determine what proteomic, transcriptomic, genomic, and metabolomic changes (i.e., molecule or molecular changes that are different in abundance in one condition or another) are specifically associated with the ability of Cef to act through MBLAC1 to reduce the actions of a substance of abuse. Network software links any such changes into functional molecular networks and/or molecular pathways. The molecules in the networks or the networks themselves can be used as biomarkers for addiction, drug response or used to develop treatments that diminish the changes seen in the networks collectively.

### Screening Identified Candidate Therapeutic Agents for Efficacy

Because Cef has been shown to have actions on cells that are nonmicrobial in nature and are linked to CNS actions of Cef, cultured cells from WT MBLAC1 and MBLAC1 KO animals (e.g., rodents), or cultured cells like those used in the experiments described in Example 1, can be used to screen (test) candidate therapeutic drugs that have one or more of the in vitro actions that Cef exerts on cell proteins, nucleic acids and metabolites. Several in vitro actions of Cef on cell proteins and metabolites have been identified, and include increasing excitatory amino acid transporter 2 (EAAT2) expression; inducing the expression of the glutamate/cystine exchanger, system x_{c}⁻; increasing mRNA expression of the specific system x_{c}⁻ subunit, xCT; increasing GSH release from cortical and spinal astrocytes; inducing nuclear factor erythroid 2-related factor 2 (Nrf2) expression; protecting cells against oxidative glutamate toxicity; etc. These actions are described in, for example, Lewerenz et al., J Neurochem. 2009 October; 111(2):332-43, incorporated by reference herein in its entirety.

Methods for screening candidate therapeutic agents that have been identified using the methods described above for addiction treatment efficacy will be apparent to one skilled in the art. The ability of a candidate therapeutic agent to treat addiction to a substance of abuse can be compared in vivo, e.g., by testing in parallel a WT MBLAC1 animal such as a rodent (e.g., a WT C57BL6/J or other rodent strain known to express MBLAC1 protein) and an MBLAC1 KO animal (e.g., rodent) made from the same strain as the WT MBLAC1 animal (e.g., rodent). Generally, the candidate therapeutic agent and the substance of abuse are administered to a WT MBLAC1 animal (e.g., rodent) and in parallel, the substance of abuse and candidate therapeutic are administered to a MBLAC1 KO animal (e.g., rodent). The animal's (e.g., rodent's) response to the substance of abuse is evaluated in the WT MBLAC1 animal and then compared to the MBLAC1 KO animal's (e.g., rodent's) response to the substance of abuse. The candidate therapeutic agent is given either once or multiple times prior to testing the sensitivity of each animal (e.g., rodent) to the substance of abuse (which could also be given once or multiple times, where physiological or behavioral actions of the substance of abuse are monitored). These actions can include changes in body temperature, blood pressure, respiration, locomotor activation, pain, seizures, willingness to act to obtain the drug (lever pressing, nosepoke, moving to area where drug is available), and physical signs of drug withdrawal. If the candidate therapeutic agent reduces or eliminates the action of the substance of abuse in the WT MBLAC1 animal but not in the MBLAC1 KO animal, the candidate therapeutic agent can be assumed to require the presence of MBLAC1 for demonstration of its anti-abuse/addiction properties.

In these methods, animals (e.g., rodents) (both KO and WT animals) can be subjected to at least one (e.g., one, two, three, four, five, etc.) appropriate drug-response (substance of abuse-response) test. Such drug-response tests are well known in the art, and are described in, for example, Abulseoud et al., Neuropsychopharmacology 39(7):1674-1684, 2014; Bell et al., Neuropharmacology 122:201-243, 2017; Philogene-Khalid et al., Behav Pharmacol. 28(6):485-488, 2017; Tallarida et al. Neurosci Lett. 556:155-159, 2013; Alajaii et al., Psychopharmacology (Berl). 228(3):419-426, 2013; Kovalevich et al., Am J Pathol. 181(6):1921-1927, 2012; I. Sondheimer & L. A. Knackstedt Behav. Brain Res. 225(1):252-258, 2011; Knackstedt et al., Biol Psychiatry 67(1):81-84, 2010; Rawls et al., Eur J Pharmacol. 584(2-3):278-284, 2008; Sari et al., J Neurosci. 29(29):9239-9243, 2009; and J. D. Steketee & P. W. Kalivas Pharmacol Rev. 2011 63(2):348-365. These references are all incorporated herein by reference in their entireties. Examples of appropriate drug-response tests include: a locomotor assay, a withdrawal assay, a sensitization assay, a self-administration assay, a reinstatement to drug assay, an analysis of white matter changes, and an analysis of changes in GLTI expression after administration of the candidate therapeutic agent and the substance of abuse.

Generally, in these methods of testing candidate therapeutic drugs in combination with substances of abuse in WT MBLAC1 and KO MBLAC1 animals (e.g., rodents), Cef is active in WT MBLAC1 animals (i.e., animals in which MBLAC1 is expressed and functional) in reducing the actions of drugs of abuse, as shown in the experiments described below involving cocaine and locomotion. In the MBLAC1 KO animals, Cef action is lost. So one can test candidate therapeutic drugs that come through a primary screen (e.g., one or more of the binding assays or the cell-based assays described herein) for ones that require MBLAC1 to act to reduce the actions of drugs of abuse.

### MBLAC1 Knock-Out Animals

In the experiments described herein, MBLAC1 KO rodents were used. However, any MBLAC1 KO animal whose Mblac1 orthologous gene is disrupted (mutated, eliminated, truncated) can be used, e.g. fish, insects (e.g., flies), worms, etc. Cef has been found to have actions on planarians (flatworms) to actions of drugs of abuse, thus planarians are an example of an MBLAC1 KO animal that can be generated and used in the methods described herein. Methods of identifying an MBLAC1 orthologue in an animal of interest are well known. See, for example, Example 1 below, which describes a bioinformatics analysis that identified MBLAC1 as the mammalian orthologue of swip-10 in C. elegans.

### MBLAC1 Knock-Out Rodents

In one embodiment of a MBLAC1 KO rodent (e.g., mouse, rat) as described herein, the MBLAC1 KO rodent has no functional copies of the Mblac1 gene (i.e., lacks both copies of the Mblac1 gene or lacks portions thereof). A MBLAC1 KO rodent as described herein includes a rodent in which the mRNA expression or mRNA translation of the Mblac1 gene has been significantly reduced by chemical or genetic means. A functional MBLAC1 KO rodent expresses full length MBLAC1 protein but has a sequence change shown to completely disable function. Herein, both types of animals are termed MBLAC1 KO. An MBLAC1 KO rodent may have had its gene modified constitutively or conditionally, e.g. at a specific stage of development or in a specific tissue or brain region. In one embodiment of an MBLAC1 KO rodent, N-terminal coding sequences of the Mblac1 gene are disrupted by CRISPR/Cas9 resulting in a complete loss of MBLAC1 protein expression in the MBLAC1 KO rodent. In one such embodiment, the Mblac1 gene includes a 5 base pair deletion that disrupts the reading frame for protein translation. A MBLAC1 KO rodent can be any type of rodent, including rats and mice. Generation and validation of the MBLAC1 KO rodent is described below in Example 2. An MBLAC1 KO rodent as described herein provides a tool to analyze the mechanisms supporting the ability of β-lactam antibiotics (e.g., Cef) to suppress cocaine actions in a mammal exposed to cocaine, and to elucidate fundamental biochemical and cellular networks that support the actions of addictive and/or therapeutic psychostimulants. Additionally, an MBLAC1 KO rodent as described herein is a useful tool for testing the ability of a candidate therapeutic agent, identified using any of the assays described herein, to treat addiction to a substance of abuse.

As described above, cells from WT MBLAC1 and MBLAC1 KO rodents can be used to test for candidate therapeutic agent specificity, in combination with the use of the WT MBLAC1 and MBLAC1 KO rodents. In one example of such an embodiment, the substance of abuse is added to the cells to evoke an in vitro response (e.g., growth, shape, metabolic rate, biochemical changes) shown to be dependent on MBLAC1 protein. Using the WT MBLAC1 and MBLAC1 KO cells tested in parallel, the user would first validate the response is present with Cef application to the WT MBLAC1 cells and absent in the MBLAC1 KO cells. Then, they could evaluate their candidate therapeutic agents on the WT MBLAC1 and MBLAC1 KO cells and look for those that show a response in the former but not the latter cells. Because cells are more high-throughput than animals, the user could consider use of this assay as a primary screen for MBLAC1-targeted therapeutic drugs and then test them for their ability to impact the actions of substances of abuse in vivo in WT MBLAC1 and MBLAC1 KO rodents. Because rodents are low throughput, they can be used as a secondary screen for in vivo activity and to relate to the in vivo actions of substances of abuse.

### Additional Methods

As mentioned above, the methods described herein are useful for identifying biomarkers for a successful therapeutic drug response that can predict efficacy during therapeutic drug development for a potential addiction treatment. Also described herein are methods of using a molecule or molecular network to predict a change in a human subject who is using a substance of abuse and responding to the substance of abuse in an MBLAC1-dependent manner, or is responding to a putative or candidate treatment in a manner similar to that seen when WT MBLAC1 animals administered a substance of abuse are treated with Cef but not when animals lacking MBLAC1 (MBLAC1 KO animals) and administered the substance of abuse are treated with Cef. These methods can be used as an assay to determine if the putative or candidate treatment is effective in a human subject and the methods of identifying molecules and networks of molecules modulated by Cef in WT MBLAC1 animals but not in MBLAC1 KO animals as described herein can identify the molecules or molecular networks that change as they should (as expected) if the putative or candidate treatment acts like Cef does in animals and in an MBLAC1-dependent manner.

### EXAMPLES

The present invention is further illustrated by the following specific examples. The examples are provided for illustration only and should not be construed as limiting the scope of the invention in any way.

### Example 1—Metallo-β-Lactamase Domain-Containing Protein 1 (MBLAC1) is a Specific, High-Affinity Target for the Glutamate Transporter Inducer Ceftriaxone

The hypothesis that MBLAC1 is an endogenous, CNS-expressed binding partner for Cef was tested using affinity chromatography and Backscattering Interferometry (BSI). As shown below, these complementary approaches demonstrate specific, high-affinity, temperature-sensitive binding between MBLAC1 and Cef in cell and brain lysates. Moreover, immunodepletion studies support MBLAC1 as a major, if not exclusive, CNS target for the antibiotic.

### Results

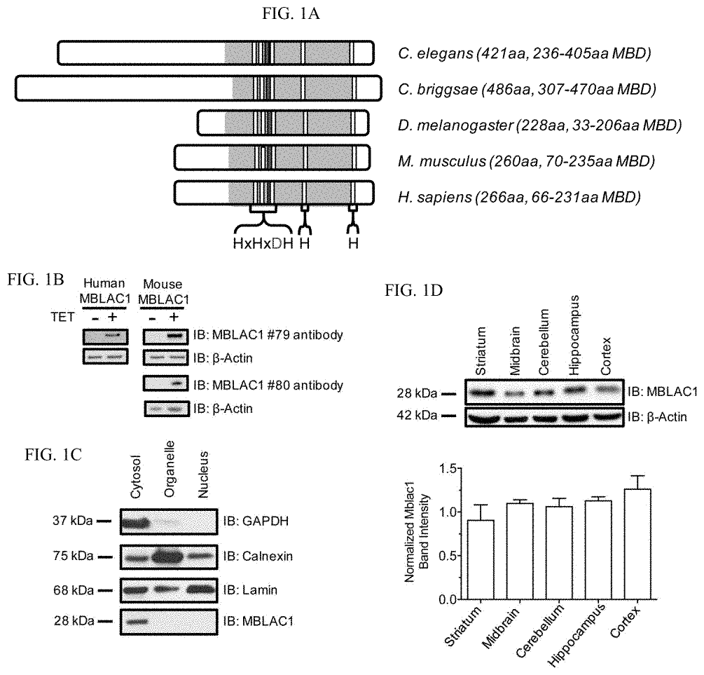

Using the proteinBLAST tool, multiple candidate SWIP-10 orthologs across phylogeny (FIG. 1A) were identified. All proteins share a single MBD that comprises the majority of the coding sequence, whereas SWIP-10 and nematode orthologs (see C. briggsae in FIG. 1A) possess a much longer N-terminus with no identified functional domains. The MBDs of each protein illustrated share His and Asp residues characteristic of metal binding and catalysis, respectively. At present, an endogenous substrate(s) for these proteins has yet to be identified.

To study the putative mouse and human orthologs of SWIP-10, MBLAC1, rabbit polyclonal antisera against MBLAC1 fusion proteins were raised and purified, and stably transfected cell lines that, in the presence of TET, express mouse or human MBLAC1 (FIG. 1B) were generated. Both antibody #79 and #80 detected mouse MBLAC1 in extracts of TET-induced HEK-cells, whereas only #79 detected human MBLAC1. Antibody #80 also detects MBLAC1 in rat brain lysates. Neither antibody detected a protein of the equivalent mass as MBLAC1 in non-induced cells.

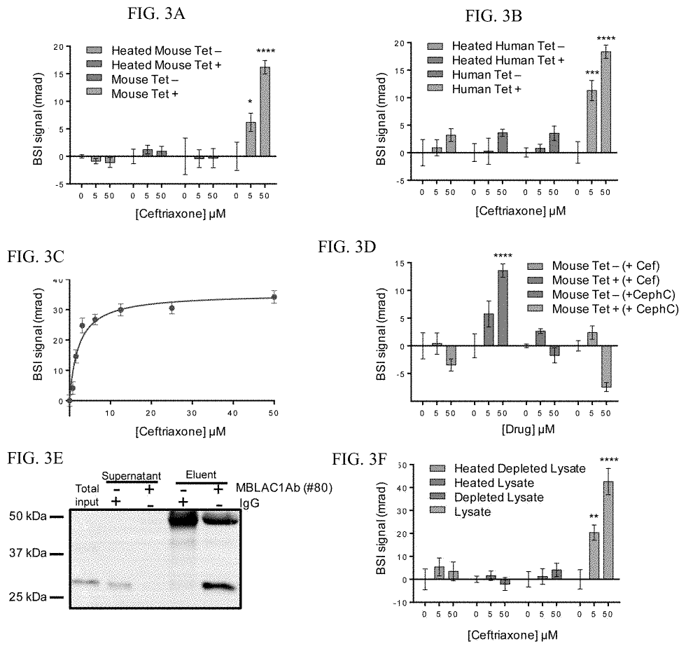

To determine the subcellular localization of endogenous MBLAC1 protein, a subcellular fractionation protocol was implemented using extracts of mouse NIH 3T3 cells (FIG. 1C). Identity of cytosolic, organelle (e.g. ER), and nuclear fractions were confirmed by immunoblotting with antibodies targeted to compartment-specific proteins. MBLAC1 protein was found to localize to cytosolic fractions characterized by glyceraldehyde 3-phosphate dehydrogenase (GAPDH) enrichment (FIG. 1C). Owing to the CNS being the likely site of action for the behavioral actions of Cef, mouse brain extracts were blotted for the presence of MBLAC1 protein. Consistent with a relatively even pattern of MBLAC1 mRNA expression detected across mouse brain regions in a prior study (Hardaway et al., The Journal of Neuroscience vol. 35, p. 9409-9423, 2015), statistically equivalent levels of anti-MBLAC1 immunoreactive protein were detected in extracts of hippocampus, striatum, cortex, cerebellum, and midbrain (FIG. 1D). Although affinity-purified antibody #80 proved suitable for detection of MBLAC1 protein in tissue extracts (see also FIG. 3E), other bands are evident on tissue western blots suggesting that immunocytochemistry pursued with this reagent is likely of insufficient specificity for evaluation of MBLAC1 regional distribution.

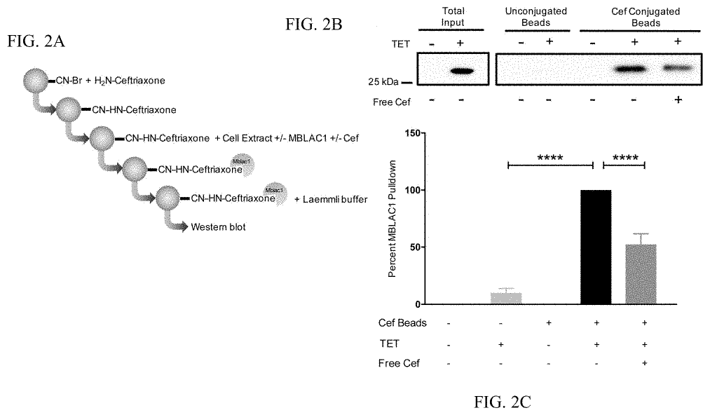

To determine if MBLAC1 and Cef interact, a MBLAC1 pulldown assay using Cef-conjugated CN—Br activated Sepharose beads was developed (FIG. 2A). Conjugated and unconjugated beads were incubated with lysates from TET or non-TET induced T-REx cells (FIG. 2B). Cef-conjugated beads extracted significantly more MBLAC1 from lysates than unconjugated beads, with no evidence of MBLAC1-immunoreactive species detected when Cef-conjugated beads were incubated with uninduced cell extracts. To further assess specificity and affinity between these two molecules, MBLAC1 expressing cell lysates were incubated with Cef (50 μM) prior to incubation with Cef-conjugated beads, which significantly diminished MBLAC1 capture (FIG. 2C).

Although the pulldown approach provided initial evidence of Cef interactions with MBLAC1, the efficiency of competition was lower than expected, based on prior studies reporting the in vitro potency of Cef for GLT-1 induction. It was reasoned that steric hindrance could limit high-affinity interactions of MBLAC1 with Cef-conjugated beads. As radiolabeled Cef was unavailable, a non-isotopic approach that could examine Cef/MBLAC1 interactions without ligand immobilization was sought. Backscattering interferometry is a sensitive approach that can detect both kinetic and equilibrium associations of unlabeled, small molecule interactions with unmodified target proteins (Bornhop et al., Science vol. 317, 1732-1736, 2007). Using lysates from uninduced and induced T-REx cells expressing mouse or human MBLAC1, BSI was implemented as described below and significant, dose-dependent BSI signals were detected only in induced cell lysates (FIG. 3A, 3B). BSI signals were eliminated by heat denaturation of extracts prior to analysis, consistent with Cef binding as arising from a proteinaceous species versus TET used to induce MBLAC1 expression.

The BSI results noted above both confirmed the Cef/MBLAC1 interactions detected in Cef-conjugated bead assays and also established a quantitative approach that could be used to estimate the affinity of unconjugated Cef for MBLAC1. Indeed, binding data collected in BSI assays conducted across a range of Cef concentrations were well fit (r2=0.96) to a single site binding equation with a K_{D} of 2.2+/−0.56 μM (FIG. 3C).

To explore the promiscuity of MBLAC1 interaction with other β-lactam antibiotics, BSI signals with cephalosporin C (CephC) were compared to those obtained with Cef. Significant dose-dependent BSI signals were again detected with Cef, but a significant CephC BSI signal (FIG. 3D) was not seen. Though these data are not statistically significant, the negative BSI signal observed in the presence of CephC indicates that there may be an interaction between MBLAC1 and CephC at high concentrations, though the nature of this interaction would appear to be molecularly different from the interaction we observe between Cef and MBLAC1. These findings further solidify the hypothesis that Cef/MBLAC1 interactions account for the reported nonmicrobial actions of Cef in the CNS.

To detect endogenous Cef binding and to determine whether MBLAC1 is likely to be responsible for observed interactions, the BSI experiments were repeated using mouse frontal cortex lysates, with and without prior heat denaturation. To determine whether, and to what degree, Cef binding signals derive from MBLAC1, BSI studies were performed on lysates that had been immunodepleted of MBLAC1 protein by anti-MBLAC1 antibody (#80). Clearance of frontal cortex lysate using MBLAC1 antibody, but not control antiserum, resulted in the elimination of MBLAC1 protein from cortical extracts (FIG. 3E). When BSI binding assays were conducted with immunodepleted extracts, the Cef binding signal was abolished (FIG. 3F). These findings support the contention that, under these binding conditions, MBLAC1 is likely the primary species in brain lysates capable of interacting with Cef at high-affinity—that the well-replicated actions Cef in the CNS arise through MBLAC1 interactions.

### Methods

### Materials and Animals

All biochemical reagents, salts and buffers were obtained from Sigma-Aldrich (St. Louis, Mo.) unless otherwise specified, and were of the highest quality available. All experiments with animals were performed under a protocol approved by the Vanderbilt Institutional Animal Care and Use Committee (IACUC). Studies with mice utilized animals of the C57BL/6J strain obtained from Jackson Laboratories (Bar Harbor, Me.).

Polyclonal Antibody Generation

Mouse brain mRNA was isolated as described and then MBLAC1 cDNA was amplified by PCR prior to cloning in frame with glutathione S-transferase (GST) in pGEX2T (GE Healthcare Life Sciences, Chicago, Ill.), followed by transformation into BL21 cells (New England Biolabs, Ipswitch, Mass.). Expression of GST-mouse MBLAC1 fusion protein was induced using 0.3 mM isopropyl β-D-1-thiogalactopyranoside (IPTG) and purified from bacterial cells via affinity chromatography using glutathione-coupled Sepharose® (GE Healthcare, Chicago, Ill.), following manufacturer's protocol. Purified GST-mouse MBLAC1 with adjuvant was injected into two rabbits (#4979 (#79) and #4980 (#80), Thermo Fisher, Waltham, Mass.) and boosted monthly to produce antiserum. To purify antisera, mouse MBLAC1 cDNA was cloned in frame with maltose binding protein (MBP) coding sequences in pMal-cRI (New England Biolabs, Ipswich, Mass.). Full length MBP-mouse MBLAC1 protein was generated as described for GST-mouse MBLAC1, with addition of 100 μM ZnSO₄ to the culture media during induction. MBP-mouse MBLAC1 fusion protein was purified via affinity chromatography over amylose resin (New England Biolabs, Ipswich, Mass. USA) and concentrated by spin filtration. To remove GST-directed antibodies, antisera were incubated with MBP-mouse MBLAC1-conjugated amylose, followed by multiple washes in column buffer (20 mM Tris-HCl, 200 mM NaCl, 1 mM EDTA, pH 7.4). Bound antibodies were eluted with 150 mM glycine pH 2.0, collecting 400 μL fractions into 2M Tris-HCl pH 8.0 for neutralization. Samples were pooled and dialyzed in 1×PBS (290 mM NaCl, 3 mM KCl, 10 mM NaHPO₄, 1.8 KH₂PO₄,) at 4° C. overnight. Dialysate was then concentrated by spin filtration and assayed for protein content (Bradford, Bio-Rad, Hercules, Calif.).

HEK Inducible Cell Line Generation

HEK Flp-In T-REx-293 cells (T-REx) expressing mouse or human MBLAC1 proteins were generated per manufacturer's (ThermoFisher, Waltham, Mass., USA) instructions. Briefly, mouse and human Mblac1 cDNAs were subcloned into pcDNA5/FRT/TO vector and then co-transfected with pOG44 plasmid (encoding Flp recombinase) by lipid based transfection into Flp-In T-REx-293 cells using TranslT-LT1 (Mirus, Madison, Wis.). Stable integrants were isolated following selection with 100 μg/ml hygromycin. Expression of MBLAC1 protein was induced by addition of 1 μg/mL tetracycline (TET) to the media. Maintenance media contained 15 μg/mL blasticidin (Life Technologies/ThermoFisher, Waltham, Mass.), 100 μg/mL hygromycin B (Life Technologies/ThermoFisher, Waltham, Mass.) as selection agents, in addition to 10% fetal bovine serum (Gibco/ThermoFisher, Waltham, Mass.), 2 mM L-glutamine, and 100 Units/mL penicillin-100 μg/mL streptomycin.

Western Blotting

Protein samples for Western blot analysis were quantified for total protein (BCA Pierce/ThermoFisher, Waltham, Mass.) and heated to 95° C. for 5 min with 1× Laemmli buffer before separation via SDS-PAGE using 10% polyacrylamide gels and transfer to Immobilon PVDF membranes (Millipore, Billerica, Mass.). Membranes were blocked for 1 hr at room temp (RT) with 5% milk in TBS/0.1% TWEEN (TBST). Primary antibody, diluted 1:1000 in 5% milk/TBST, was incubated with membranes overnight at 4° C. After washing 4× for 5 min with TBST, secondary antibody (peroxidase-conjugated mouse-anti-rabbit, Jackson ImmunoResearch, West Grove, Pa.) in 5% milk/TBST was incubated for 1 hr at RT. Blots were washed again before band visualization and quantitation by enhanced chemiluminescence (BioRad Clarity ECL, Hecules, Calif.) using an ImageQuant LAS 4000 imager (GE Heathcare Life Sciences, Chicago, Ill.).

Subcellular Fractionation

Plated 3T3 cells were washed in PBS and pelleted for resuspension in a digitonin buffer (150 mM NaCl, 50 mM HEPES, 200 μg/mL digitonin) for 10 min while rotating. Lysate was then spun at 2000×g and resultant supernatant was kept (cytosolic fraction). The remaining pellet was resuspended in an NP40 buffer (150 mM NaCl, 50 mM HEPES, 1% NP40) and lysate was left on ice in NP40 buffer for 30 min and then centrifuged at 7000×g. Resultant supernatant was kept for membrane and organelle fraction, whereas the pellet was resuspended in RIPA buffer (150 mM NaCl, 50 mM HEPES, 0.5% Na-deoxycholate, 0.1% SDS, 1 U/ml Benzonase) and rotated for 1 hr at 4° C. then centrifuged for 10 min at 7000×g. Supernatant was kept for nuclear protein fraction. Each fraction was then subjected to Western blot analysis as described above.

Immunodepletion Studies

Wild type C57BL/6J mice (Jackson Labs, Bar Harbor, Me.) were rapidly decapitated and frontal cortex was dissected. Tissue was homogenized in ice cold 20 mM HEPES buffer (pH 7.4) using a Dounce homogenizer (Wheaton, Millville, N.J.), and then sonicated (F60 sonic dismembrator, Fisher Scientific, Waltham, Mass.) using 5, 1 sec pulses. Lysates were then centrifuged at 100,000×g for 30 min. Supernatants were collected, diluted with 2× lysis buffer (40 mM HEPES, 220 mM KCl, 20 mM NaCl, 4 mM MgCl, 10 mM KH₂PO₄, 500 μM ZnSO₄) and protein concentration determined (BCA Protein Assay, ThermoFisher, Waltham, Mass.). Lysates were incubated at 4° C. overnight with 5 μg of either rabbit IgG (Antibodies Inc., Davis, Calif.) or affinity-purified MBLAC1 antibody #80. Samples were then incubated with Magnetic Protein G beads (Dynabeads, ThermoFisher, Waltham, Mass.) for 2 hr at 4° C. Supernatants were removed for BSI and Western blot analysis. A portion of supernatants were heat denatured at 95° C. for 5 min prior to BSI experiments (see below). Beads were washed 3× with 1× lysis buffer and MBLAC1 protein was eluted with 4× Laemmli buffer and diluted prior to SDS PAGE and Western blot analysis.

Affinity Capture of MBLAC1 with Cef-Conjugated Sepharose®

Cyanogen Bromide (CN—Br) Activated Sepharose® 4B beads were prepared and conjugated to Cef following manufacturer's recommendations (GE Healthcare Life Sciences, Pittsburgh, Pa.). Briefly, lyophilized beads were suspended and washed 3× in 1 mM HCl, pH 3.0. Beads were then equilibrated in coupling buffer (500 mM NaCl, 0.1 mM NaCHO₃, pH 8.3), and divided into conjugated and unconjugated samples. Conjugated beads (250 μL) were left to rotate for 1 hr at room temp with 2.5 μmoles of Cef in coupling buffer. The unconjugated beads were treated the same except no Cef was added. After coupling, remaining reactive CN—Br groups were inactivated by incubation (3 hr) with 0.1M Tris-HCl (pH 8.0). Following inactivation, beads were subjected to alternating acid/base washes with 0.1M Na-acetate (pH 4.0) and 0.1M Tris-HCl (pH 8.0). Beads were then equilibrated in lysate buffer prior to lysate addition. Final protein concentrations (100 μg protein at 0.1 mg/mL) were incubated for 1 hr at room temp with 50 μL beads (50% slurry). In competition experiments, excess Cef (50 μM, final concentration) was mixed with lysates 10 min prior to addition of beads at room temp. Beads were washed 5× in lysis buffer prior to elution of bound MBLAC1 in 2× Laemmli buffer, followed by SDS-PAGE and Western blot analysis.

Backscattering Interferometry Binding Assays

To assess the binding of unlabeled Cef with MBLAC1 in cell and tissue lysates, BSI was utilized. A BSI instrument was assembled and employed (Kussrow et al., Anal Chem 84, 779-792, 2012). Briefly, BSI, using a semi-circular chip, a helium-neon laser, and CCD camera, allows for high-sensitivity, refractive index (RI) sensing. The RI signal is obtained by isolating shifts in the backscattered fringes by Fourier analysis. RI changes are quantified by comparing fringe shifts between test and reference samples.

Lysates for BSI contained Cef at concentrations of 0 μM, 5 μM, and 50 μM and concentrations of either 300 μg/mL (mouse cell lysates) or 100 μg/mL (human cell lysates and mouse brain lysates) protein. Samples were incubated on ice for 1 hr prior to analysis to favor equilibrium binding conditions. To obtain BSI signals, the sample with 0 μM Cef was injected into the channel in a stop-flow manner using a vacuum and allowed to reach temperature and pressure equilibrium (˜10 sec) at which point the phase value (BSI signal) was measured for 20 sec. Specific BSI signal was determined by subtracting Cef only signal from Cef-lysate signals. To determine binding affinity, BSI was conducted as above using Cef concentrations ranging from 0.78 μM to 50 μM. Signals were plotted versus concentration and fitted with a single-site saturation binding curve using Prism 6.0 software (GraphPad, Inc., La Jolla, Calif.).

### Example 2—Global Untargeted Serum Metabolomic Analyses Nominate Metabolic Pathways Responsive to Loss of Expression of the Orphan Metallo β-Lactamase, MBLAC1

Above in Example 1 it was shown that MBLAC1 is a specific, and possibly exclusive, high-affinity target for the β-lactam antibiotic, Cef. Multiple studies reveal that Cef can elevate glial expression of plasma membrane Glu transporters that can normalize pathologically altered extracellular Glu levels. However, neither the endogenous substrate nor an ascribed metabolic pathway, have been established for MBLAC1, though the ability of Cef to afford neuroprotection against Glu related pathology in many brain disorders and block reinstatement to drugs of abuse after withdrawal, suggests that advances in substrate and pathway elucidation may be of clinical significance. Although there is significant functional information in worms concerning the cellular and physiological impact of swip-10 mutations, the gene is expressed in a small number of cells, making a biochemical comparison between wildtype and mutant strains problematic. In contrast, the murine Mblac1 gene is widely expressed. Thus, in these experiments, biochemical differences between wildtype (WT) and Mblac1 knockout (KO) mice were characterized, the KO mice produced using a CRISPR/Cas9 approach. Here both the successful generation of viable Mblac1 KO mice and efforts to use these animals to investigate the in vivo biochemical impact of loss of MBLAC1 expression are reported. These results demonstrate the use of the KO mice for demonstrating specific pathways that can be activated or suppressed by loss of MBLAC1 and demonstrate how one can use the KO mice to look for specific pathways that are dependent on MBLAC1 expression

Here the results of these efforts to interrogate the serum metabolome of MBLAC1 KO and age-matched WT mice are reported. To resolve serum small molecules responsive to loss of MBLAC1 expression, an ultra-performance liquid chromatography coupled to mass spectrometry (UPLC-MS/MS)-based analysis was implemented. Reported are the presence of unique biosignatures that distinguish the sera of MBLAC1 KO from WT mice, with replicated, over-representation of features linked to primary bile acid biosynthesis and linoleate metabolism. These networks are discussed in the context of the biology of the MBLAC1 ortholog SWIP-10, as well as the neuroprotective actions of chronic Cef administration.

### Methods and Materials

Generation of MBLAC1 KO Mice

Initial untargeted metabolomics experiments and generation of the MBLAC1 KO mice were performed under a protocol approved and annually reviewed by the Vanderbilt Institutional Animal Care and Use Committee. For a subsequent pathway validation metabolomic study, experiments were performed under a protocol approved and annually reviewed by the Florida Atlantic University Institutional Animal Care and Use Committee. In all experiments, mice were housed on a 12:12 LD cycle with food and water available ad libitum. To implement a CRISPR/Cas9 based strategy for producing MBLAC1 KO mice, software developed in the Zhang laboratory (Massachusetts Institute of Technology) was utilized to evaluate sequences in the first exon, where we identified an optimal protospacer adjacent motif (PAM) sequence located 43-45 bp 3′ of the ATG start site. A guide RNA was generated with sequence that matched the protospacer adjacent to the PAM—3′ to 5′: GGAAACGACCGCAGGTCGCCG (SEQ ID NO:3) (PAM site underlined). Sense and antisense oligonucleotides (Sigma Aldrich, St. Louis, Mo.) encoding the guide RNA were annealed and inserted into the plasmid pX330 (Addgene plasmid #42230) which also encodes CAS9 (Cong et al., Science 2013 DOI: science. 1231143 [pii] 10.1126/science. 1231143). Injection of the plasmid into C57BL6/J embryos was performed in the Vanderbilt ES/Transgenic Mouse Core. From these injections one male pup was identified as having a 5 bp deletion at the targeted site, deleting bp 46-50, and another male pup was identified as having a 14 bp deletion at the targeted site, deleting bp 44-57, as verified by Sanger sequencing (Genewiz). KO mice referred to in the present study represent progeny of the 5 bp deletion founder. Genotyping of MBLAC1 KO mice was performed by TransnetYX, Inc (Cordova, Tenn., USA) using separate PCR reactions to genotype for WT (forward primer: GACAGCGATAGTTTAGTTTC (SEQ ID NO: 4), and reverse primer: TTGCTGGCGTCCAGCGGC) (SEQ ID NO:5), 5 bp deletion MBLAC1 KO (forward primer: GACAGCGATAGTTTAGTTTC (SEQ ID NO: 6) and reverse primer: TCCCTGGCGTCCAGCGGC) (SEQ ID NO:7) and 14 bp deletion MBLAC1 KO (forward primer: CGAGCCCCTGCATCCT (SEQ ID NO: 8) and reverse primer: GCCGCGCAGCAGAAC) (SEQ ID NO:9). KO mice were mated with WT C57BL6/J females and heterozygous KO pups were outcrossed to C57BL6/J mice for 3 additional generations to limit the presence of off-target mutations in mice used for analysis.

Evaluation of MBLAC1 Protein Expression by Western Blotting

All chemicals used in tissue homogenization and immunoblotting assays, unless otherwise specified, were obtained from Sigma-Aldrich (St. Louis, Mo., USA). For western blots to validate loss of MBLAC1 protein, male mice were killed by rapid decapitation and whole brains were removed to an ice-cold metal plate and dissected into specific regions. Freshly dissected brain regions were homogenized in RIPA buffer (50 mM Tris, pH 7.4, 150 mM NaCl, 1 mM EDTA, 1% TRITON X-100, 1% sodium deoxycholate, 0.1% SDS) with a Dounce homogenizer and then solubilized for 1 hr at 4° C. while rotating. Protein lysates were centrifuged at 4° C. for 30 min at 15,000×g to remove insoluble material. Protein concentrations of supernatants were determined using the BCA method (ThermoFisher, Waltham, Mass., USA) and 40 μg of brain (cortical tissue) protein and 60 μg of liver protein was separated by 10% SDS-PAGE, transferred to PVDF membranes (Miillipore Sigma, Billerica, Mass., USA). Membranes were blocked using 5% dry milk in TBS/0.1% Tween (TBST) for 1 hr at room temperature (RT) prior to incubation with affinity-purified MBLAC1 #4980 antibody (1:1000 dilution in 5% milk with TBST—incubated overnight at 4° C. followed by 4×5 minutes with TBST). HRP-conjugated, mouse anti-rabbit secondary antibody (Jackson ImmunoResearch, West Grove, Pa.) was used at 1:10000 dilution. B-Actin was detected using a 1:20,000 dilution of β-actin-HRP antibody (Sigma-Aldrich, St. Louis, Mo.). Immuno-reactive bands were identified by chemiluminescence (Clarity, BioRad, Hercules, Calif., USA) and imaged with an LAS4000 imager (GE Healthcare Life Sciences, Pittsburgh, Pa., USA) and analyzed with associated ImageQuant™ software (GE Healthcare Life Sciences, Pittsburgh, Pa., USA).

Serum Sample Preparation

The initial untargeted study made use of serum collected from three, age- (12-16 wks) and sex- (female) matched WT and KO mice. WT mice were commercially obtained C57BL/6J mice (Jackson Labs, Bar Harbor Me., USA). The subsequent pathway validation study reported is derived from serum collected from four sex-(female) matched WT and KO littermates (aged 12-16 weeks) bred from MBLAC1 heterozygous parents. Following rapid decapitation of mice, 0.5-0.75 mL of trunk blood (blood immediately collected from the body at the site of decapitation) was collected, allowed to coagulate on ice for 30 min and centrifuged (15 min at 5,000 rpm). Serum (50 μL) was collected into fresh tubes followed by addition of ice cold 80% methanol (5× by volume), then stored at −80° C. overnight. On the next day, samples were centrifuged at 10,000 rpm for 15 min to eliminate methanol precipitated proteins. This methanol precipitation step was repeated and the metabolite containing supernatant was dried via speed-vacuum and stored at −80° C. until analysis.

Global, Untargeted UPLC-MS/MS Analysis

For mass spectrometry analysis, dried extracts were reconstituted in 100 μL of acetonitrile/water (80:20, v/v) and centrifuged for 5 min at 15,000 rpm to remove insoluble material. Quality control (QC) samples were prepared by pooling equal volumes from each experimental sample. Full MS (FMS) data was acquired for this QC pool, in both HILIC-POS (3 FMS QC runs) and HILIC-NEG (1 FMS QC runs) methods, to use as a retention time alignment reference within Progenesis QI for subsequent normalization and data quantitation. MS/MS (data dependent (DD)) acquisitions for pooled QCs were run to assess instrument performance over time and used for feature annotation (described below).

MS analyses were performed on a Q-Exactive HF hybrid mass spectrometer (Thermo Fisher Scientific, Bremen, Germany) equipped with a Vanquish UHPLC binary system and autosampler (Thermo Fisher Scientific, Germany). Extracts (5 uL injection volume) were separated on a SeQuant ZIC-HILIC 3.5-μm, 2.1 mm×100 mm column (Millipore Corporation, Darmstadt, Germany) held at 40° C. Liquid chromatography was performed at a 200 μL min-1 using solvent A (5 mM ammonium formate in 90% water, 10% acetonitrile) and solvent B (5 mM ammonium formate in 90% acetonitrile, 10% water) with the following gradient: 90% B for 2 min, 90-40% B over 16 min, 40% B held 2 min, and 40-90% B over 10 min, 90% B held 10 min (gradient length 40 min). Full MS analyses were acquired over a mass range of m/z 70-1050 under an ESI positive profile mode and separately under an ESI negative profile mode. Full mass scan was used at a resolution of 120,000 with a scan rate at ˜3.5 Hz. The automatic gain control (AGC) target was set at 1×106 ions, and maximum ion injection time (IT) was at 100 ms. Source ionization parameters were optimized with the spray voltage at 3.0 kV, and other parameters were as follows: transfer temperature at 280° C.; S-Lens level at 40; heater temperature at 325° C.; Sheath gas at 40, Aux gas at 10, and sweep gas flow at 1. Data dependent (DD) MS/MS spectra were acquired using a data dependent scanning mode in which one full MS scan (m/z 70-1050) was followed by 2 MS/MS scans. MS/MS scans are acquired in profile mode using an isolation width of 1.3 m/z, stepped collision energy (NCE 20, 40, 60), and a dynamic exclusion of 6 s. MS/MS spectra were collected at a resolution of 15,000 with an AGC target set at 2×105 ions, and IT of 100 ms. To assess instrument performance and reproducibility throughout our experimental run sequence, the retention times and peak areas were determined for a subset of identified endogenous molecules (n=10) observed in the 3 DD QC pool runs bracketing the experimental FMS QC and experimental run sequence (visualized using Skyline (MacLean et al., Bioinformatics vol. 26, p. 966-968, 2010). These data demonstrate the reliability of the UPLC-MS/MS platform minimizing the importance of technical replicates.

Metabolite Data Processing and Analysis

UPLC-MS/MS raw data were imported, processed, normalized, and reviewed using Progenesis QI v.2.1 (Non-linear Dynamics, Newcastle, UK). All FMS sample runs were aligned against a FMS QC pool reference, with alignment to the reference being ≥97%, demonstrating the reproducibility of the HILIC column separation method. Peak picking, with a minimum threshold of 250,000 ion intensity, was performed for individual aligned runs based on an aggregate run (representative of all ion peaks detected in all samples). Unique ions (retention time and m/z pairs) were grouped (a sum of the abundancies of unique ions) using both adduct and isotope deconvolutions to generate unique “features” (retention time and m/z pairs) representative of unannotated metabolites. Data were normalized to all features using Progenesis QI. Briefly, all runs have a measurement for every feature ion, therefore a ratio can be taken for the feature ion abundance in a particular run relative to the value in the normalization reference. Progenesis applies a Log 10 transformation to the ratio to yield a normal distribution on all ratio data within each run for all samples, and scalar estimations shift the Log 10 distributions onto that of the normalization reference. Resulting FMS data was utilized for relative quantitation. The minimum percent coefficient of variance (% CV) was determined for all features across sample groups. Data was exported to EZ Info (Umetrics Software) and unsupervised (% of mean) Principle Components Analysis (PCA) was used to visualize clustering of data groups (all features included) prior to statistical tests of significance. Additionally, within Progenesis QI, a one-way analysis of variance (ANOVA) test was used to assess significance between WT and KO groups and returned a P-value for each feature (retention time_m/z descriptor), with a nominal P-value ≤0.05 taken as significant. Significant features were further filtered using a fold change threshold calculated by Progenesis from combined abundance data, with a cutoff of FC≥|1.2| deemed as significant. Multiple testing correction (MTC) was conducted with Bioconductor's q-value package using the Storey method with the π0 method set to “bootstrap”, a false discovery rate (FDR) level ≤0.1, and default parameters. Visualizations of dysregulated metabolites were represented by volcano plots (log 2 (fold change) vs. −log 10 (P-value)). Tentative and putative annotations were determined within Progenesis using accurate mass measurements (<5 ppm error), isotope distribution similarity, and manual assessment of fragmentation spectrum matching (when applicable) from the Human Metabolome Database (HMDB), Metlin, MassBank, and the National Institute of Standards and Technology (NIST) database. Additional putative annotations were assigned using Compound Discoverer 2.0 (Thermo Scientific, Waltham, Mass., USA). Briefly, the DDA data was uploaded to Compound Discoverer 2.0, deconvoluted to group isotopes/adducts of the same feature, and features were assigned an m/z Cloud spectral match score based on feature spectral matches against the mzCloud spectral libraries. For Level 3 confidence features (i.e., annotations supported by MS1 level data that may match multiple candidate annotations, including potential isomeric matches with indistinguishable chemical formula and spectral matches), mummichog 2.0 (Li et al., PLoS Comput Biol vol. 9, e1003123, 2013) was utilized to rank the most likely species within the samples. mummichog 2.0 predicts biological activity from MS1 data rather than formal manual curation of MS-2-dependent identifications. The MetaboAnalyst 3.0 program was used for pathway and metabolite set enrichment analyses using the list of statistical significance annotated features in the discovery dataset. KEGG metabolite pathways were visualized using Cytoscape 3.4.0 (The Cytoscape Consortium, USA). Increased confidence in the annotation of many features was achieved by manually assessing spectral match and RT consistencies between experimental data and chemical standards within a curated in-house library. Chemical standards (purchased from Sigma Aldrich (St. Louis, Mo.) unless otherwise specified) were prepared at a concentration of 10 ng/uL in acetonitrile/water (80/20, v/v).

Validation of Pathway Disruptions Via Metabolomic UPLC-MS/MS Analysis

UPLC-MS/MS raw data were imported, processed, normalized, and reviewed using Progenesis QI v.2.1 as described above for the initial discovery dataset with an additional pooled QC DD run acquired in the middle the sample injection sequence. After the raw data was imported and processed in Progenesis, mummichog 2.0 was used to perform pathway enrichment analysis by predicting biological activity from MS1 data allowing a focused assessment and validation of specific pathways sensitive to MBLAC1 KO. Significant pathways were determined using the Fisher exact test and corrected P-values were determined by modeling the raw P-values as a Gamma distribution and adjusted on the cumulative distribution function (CDF) of the Gamma model.

### Results

Generation and Validation of MBLAC1 KO Mice.

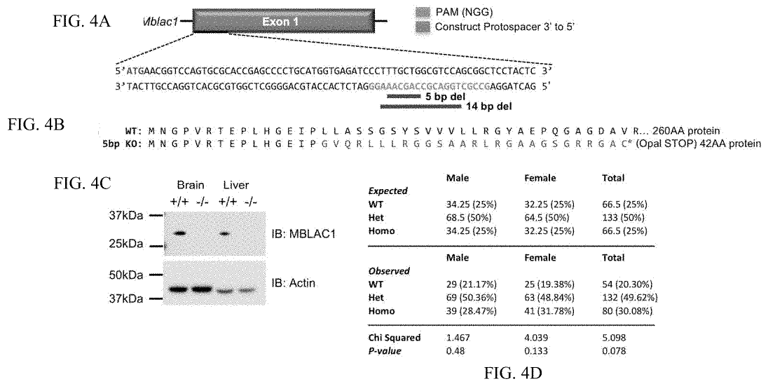

To eliminate expression of MBLAC1 in vivo and initiate a metabolomic interrogation of MBLAC1-linked pathways, a non-homologous end joining (NHEJ) CRISPR/Cas9 strategy was used to introduce deletions in the Mblac1 gene, disrupting sequences that encode the N-terminus of MBLAC1 protein as described in the Methods above (Hsu et al., Nat Biotechnol vol. 31, 827-832, 2013; Shen et al., Cell Res vol. 23, 720-723, 2013). This effort yielded two different deletion lines with either 5 bp or 14 bp deletions. The studies described in this report, derive solely from experiments with mice that harbor the 5 bp deletion, which lies 46 bp downstream of the MBLAC1 protein start site (FIG. 4A). The resulting frame shift results in the generation of 27 amino acids of ectopic sequence prior to strand termination (FIG. 4B). As shown in FIG. 4C, immunoblots of brain (cortical tissue) and liver extracts prepared from 5 bp deletion-containing KO mice, using affinity-purified MBLAC1 antibody, demonstrated complete loss of the 27 kDa band predicted to encode MBLAC1 protein (FIG. 4C) (see Example 1 above). The founder mouse, as well as subsequent heterozygous and homozygous KO progeny, were viable, produced offspring at normal Mendelian ratios (FIG. 4D), and exhibited no visible physical or behavioral abnormalities.

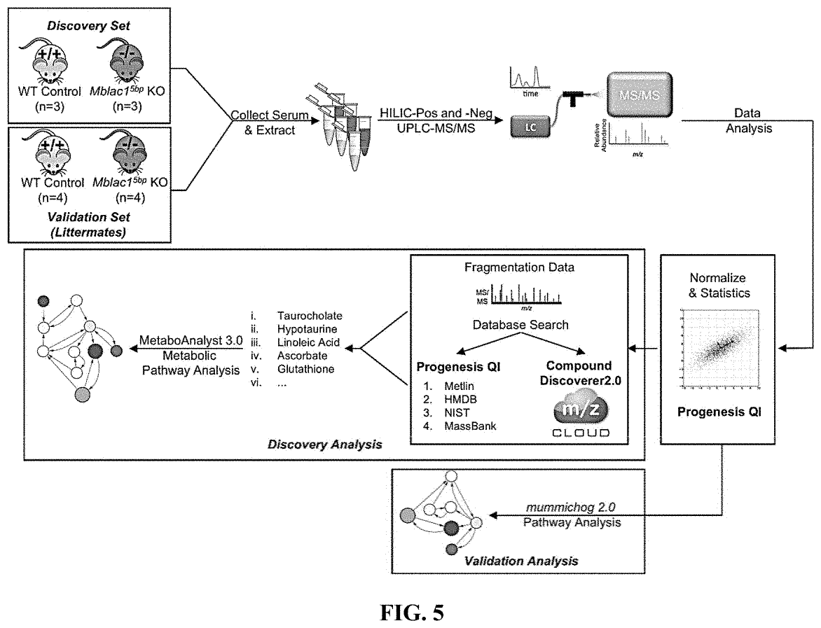

The experimental design, from serum collection through data analysis, is depicted in FIG. 5. Serum samples were collected from WT and MBLAC1 KO mice and metabolites were separated by polarity using HILIC-POS and -Neg UPLC-MS/MS. For confidence in metabolite detection and putative identification of features, two complementary data processing and analysis platforms were pursued, Progenesis QI and Compound Discoverer 2.0 as described in Methods. Briefly, Progenesis QI was used for peak-picking, normalization and statistical analysis to determine uniqueness of MBLAC1 KO and WT sera metabolomes. Both Progenesis QI and Compound Discoverer 2.0 were used to assign annotations to features of interest based on database searches and spectral library matching. The compiled list of annotated, significantly regulated features was subsequently analyzed by MetaboAnalyst 3.0. where enrichment of known metabolic pathways was assessed. This approach was designed to identify metabolic pathways affected by loss of MBLAC1 expression, and thereby provide a physiological context for contributions of MBLAC1 substrate(s).

Elucidation of an MBLAC1-dependent Serum Metabolome

UPLC-MS/MS methods are now commonly used for metabolomic studies owing to their high-resolution and sensitivity capabilities (Lin et al., J Proteome Res vol. 10, p. 1396-1405, 2011). As many endogenous metabolites found in serum samples are expected to be polar/hydrophilic, efforts were initiated using HILIC to retain and resolve polar analytes. Both HILIC-positive (POS) ion mode and HILIC-negative (NEG) ion mode MS methods were used to increase the molecular breadth of detected metabolites. Future studies may benefit from complementary reverse-phase liquid chromatography (RPLC)-MS methods. Representative total ion chromatograms for serum samples derived from WT and KO mice were produced. The Progenesis QI data processing platform was used, to inspect these runs for reproducible, genotype-dependent differences by normalizing to all feature abundances (each feature abundance is a sum of feature ion abundances comprised of grouped adduct forms). While not a direct indicator of efficacy, these analyses detected many molecular features (with unique mass to charge ratios (m/z)) in the data set, 2002 features in HILIC-POS and 2336 features in HILIC-NEG. Within Progenesis QI, feature sample variance is defined by the minimum percent coefficient of variance (min % CV) from any experimental group such that a low % CV value represents less abundance variance among biological samples. Based on other untargeted metabolomic studies, features with a min % CV≤30% were considered as having acceptable abundance variation, with 69% of the features in HILIC-POS have a min % CV≤30% and 57% of the features in HILIC-NEG have a min % CV≤30%. The binning of features by min % CV ranges was determined. Subsequent, unsupervised PCA of these data revealed clear and consistent segregation of WT and KO biological replicates distinct from the pattern of pooled reference samples.

Next, a one-way ANOVA was used to nominate features that demonstrated genotype-dependent abundance differences between WT and KO samples, with a nominal P-value of ≤0.05 taken as significant. For HILIC-POS data, ANOVA analysis revealed 326 features as significant, 16% of the total number of features. For samples analyzed by HILIC-NEG, 287 features, 12% of the total, reached significance. In these discovery experiments, a liberal fold change [(FC) ≥|1.2|] was used as the filtering threshold, based on previous plasma metabolomics studies. Features significantly dysregulated between WT and KO samples from HILIC-POS and -NEG respectively were determined.

Nomination of Biomarkers of Loss of MBLAC1 Expression.

Metabolite identification was pursued for significant features, with a nominal P-value ≤0.05 and a FC ≥|1.2|. The experimental m/z measurement of each feature was queried against several published metabolite databases (i.e., HMDB, MassBank, Metlin, NIST, mzCloud) to match feature m/z within a ±5 ppm window. Various levels of confidence were assigned to the metabolite annotations (Table 1) based on the levels of metabolite identification first outlined by Sumner et al. 2007 and the Metabolomics Standard Initiative (Sumner et al., Metabolomics: Official Journal of the Metabolomic Society vol. 3, 211-221, 2007) and the more recent adaptations of this approach (representative suggested tentatively/putatively annotated features significantly sensitive to MBLAC1 loss from a discovery dataset). Several of the prioritized molecules do not match any current database entries, either representing novel metabolites (unknown unknowns) or unknown degradation or breakdown products that are absent from existing databases. These are classified most broadly as level 5 (L5) for a feature annotated with a unique m/z. A subset of the significantly regulated molecules in the data, classified as level 4 (L4), could be assigned multiple potential molecular formulas and thus render multiple candidate annotations. Level 3 (L3) features are classified based on a confident molecular formula and accurate mass. Tentative identifications were assigned to many L3 features by using mummichog 2.0 to predict the species found in the samples, and these putative annotations were denoted. Features are classified as level 2 (L2) when experimental fragmentation data is consistent with a spectral library match upon manual assessment and curation, rendering a putative. Pure reference standards generate match scores ranging from 20/100 to >99/100 against external spectral libraries. Thus, an arbitrary threshold of 45/100 was set to facilitate curation. A lower fragmentation score match was accepted for features with a low (<100) m/z that matched a single metabolite, in which case the low fragmentation score is likely a result of minimal fragmentation as well as potential MS/MS fragments being below the detection limit of the instrumentation platform. Together, Progenesis QI and Compound Discoverer 2.0 facilitated annotations for 16% (92 out of 593) of the significantly different features. The highest identification, confidence level (L), is achieved by comparison of experimental data with that of a standard reference compound to confirm the structure with retention time, isotope pattern, and fragmentation.

TABLE 1

| | Initial Untargeted UPLC-MS/MS | | | |
|---|---|---|---|---|
| | | | | Confidence |
| Pathway | Name | Formula | Mol. Wt. | level |
| Taurine and | Pyruvic acid** | C3H4O3 | 88.0160 | L3 |
| hypotaurine | L-alanine | C3H7NO2 | 89.0477 | L2 |
| metabolism | Taurine | C2H7NO3S | 125.0146 | L2 |
| | Hypotaurine [symbol] | C2H7NO2S | 109.0197 | L1 |
| | 3-Sulfinoalanine | C3H7NO4S | 153.0096 | L2 |
| | Taurohyocholic acid*/Taurocholic acid* [symbol] | C26H45NO7S | 515.2917 | L3 |
| | 2-Hydroxyethanesulfonate [symbol] | C2H6O4S | 125.9980 | L2 |
| Primary bile | Glycine | C2H5NO2 | 75.0320 | L1 |
| acid | Taurine | C2H7NO3S | 125.0144 | L2 |
| biosynthesis | Cholic acid | C24H40O5 | 408.2880 | L2 |
| | Chenodeoxycholic acid*/Deoxycholic acid* | C24H40O4 | 392.2927 | L3 |
| | Chenodeoxycholic acid*/Deoxycholic acid* [symbol] | C24H40O4 | 392.2927 | L3 |
| | Taurohyocholic acid*/Taurocholic acid* [symbol] | C26H45NO7S | 515.2917 | L3 |
| | Taurochenodeoxycholic acid [symbol] | C26H45NO6S | 499.2967 | L2 |
| Glutathione | L-glutamate | C5H9NO4 | 147.0532 | L1 |
| Metabolism | Glycine | C2H5NO2 | 75.0320 | L1 |
| | Ascorbic acid** [symbol] | C6H8O6 | 176.0321 | L2 |
| | Ornithine | C5H12N2O2 | 132.0899 | L2 |
| | gamma-L-Glutamyl-L-cysteine** | C8H14N2O5S | 250.0623 | L3 |
| | Pyroglutamic acid [symbol] | C5H7NO3 | 129.0426 | L2 |
| | Dehydroascorbic acid** [symbol] | C6H6O6 | 174.0164 | L3 |
| Linoleic | Linoleic acid [symbol] | C18H32O2 | 280.2402 | L2 |
| acid | 13(S)-HpODE | C18H32O4 | 312.0230 | L2 |
| metabolism | 13(S)-HODE*/9(10)-EpOME* | C18H32O3 | 296.2347 | L3 |
| | 13-OxoODE** [symbol] | C18H30O3 | 294.2195 | L3 |
| | 13(S)-HODE*/9(10)-EpOME* | C18H32O3 | 296.2347 | L3 |

*Isomeric metabolites cannot be differentiated in our data by MS2 or RT, thus both potential candidates are indicated and denoted as L3.
**L3 confidence level indicates that a feature has multiple candidate identification. Mummichog 2.0 was used to rank the most likely species which is denoted in table.
Metabolites of the identified pathways of interested to be confirmed and utilized for a future targeted MBLAC1 KO metabolomics studies. ID levels for each listed metabolite is based on the degree of confidence of putative identification (based on database identification and fragmentation data supporting ID) described in Sumner et al., 200745 and Schrimpe-Rutledge et al., 2016.33.
Those indicated with a downward arrow were downregulated in MBLAC1 KO mice, and those indicated with an upward arrow were upregulated in MBLAC1 KO mice.

Nomination of MBLAC1-Dependent Metabolic Pathways

To identify metabolic pathways altered by MBLAC1 KO, analysis with features of interest exhibiting moderate to high confidence levels of identification (L3-L1) was pursued. MetaboAnalyst 3.0 was used to map the 92 significantly dysregulated, putatively-identified metabolites to Kyoto Encyclopedia of Genes and Genomes (KEGG) defined pathways. After identifying the most dysregulated pathways, the total coverage of each pathway that was identified in the dataset was determined which allowed the increase of confidence in KEGG pathway assignment. HILIC-MS/MS provides effective retention, separation, and elution of polar molecules and consequently, lower representation of non-polar molecules is expected, and thus one would not expect to obtain full coverage of metabolic pathways. Several pathways, however, were identified as warranting further inspection, including taurine and hypotaurine metabolism, primary bile acid biosynthesis, glutathione metabolism, and linoleate metabolism.

The KEGG defined pathway for taurine and hypotaurine metabolism overlaps at multiple points with the pathway supporting primary bile acid homeostasis. The pathway intersection (containing 31 metabolites) is highlighted in a user-defined, hybrid “taurine, hypotaurine and primary bile acid metabolism” pathway with the highest (68%) coverage of metabolites in the dataset. Furthermore, 16% of the metabolites (i.e., 5 features) in this combined pathway are putatively identified as significantly reduced in KO samples (Table 1) with large fold changes (i.e. Taurochenodeoxycholic acid FC=|49.1|) observed, underscoring these pathways as particularly sensitive to the absence of MBLAC1 expression. Furthermore, the two linked pathways noted can also be associated with glutathione (GSH) metabolism. Thus, although no change was observed in cysteine, this amino acid is a key precursor to the synthesis of taurine related metabolites and is also a key amino acid in the GSH pathway, which MetaboAnalyst 3.0 KEGG pathway analysis revealed to be significantly impacted by loss of MBLAC1 expression, with 8% (3 features) of KEGG GSH metabolites altered in KO serum (Table 1). Lastly, the MetaboAnalyst 3.0 KEGG pathway analysis identified linoleate metabolism as a pathway with changes in a sizeable number of metabolites detected (40% total metabolic pathway coverage and identified to have 13% over-representation of significantly dysregulated metabolites). Together these findings encouraged a follow up experiment of MBLAC1 KO metabolic changes, in comparison to MBLAC1 WT, to validate the impact of the MBLAC1 KO, with particular reference to the metabolic pathways highlighted above (pathways of interest).

Validation of Metabolic Pathway Disruptions Induced by Loss of MBLAC1

Using an independent set of serum samples prepared from four age- and sex-matched (female) littermate MBLAC1 KO and four WT mice, follow-up metabolic pathway based analyses were conducted to provide preliminary validation of MBLAC1 sensitive metabolic pathways determined from the initial age and sex-matched, but non-littermate derived serum samples (FIG. 5). The validation dataset corroborated the presence of 80% (19/24) of the unique features putatively identified in pathways of interest (Table 1) in the discovery set of serum samples by Progenesis QI, though some features were not detected. Utilizing the second set of serum samples to pursue validation of the discovery dataset at the specific pathway level, mummichog 2.0, was again used to determine the metabolic pathways impacted by loss of MBLAC1 (FIG. 5). The software predicted bile acid biosynthesis (P-value=0.042, 5 significant features out of 18 pathway features) and linoleate metabolism (P-value=0.0002, 7 significant features out of 14 pathway features), reproducing two of the pathways from the initial discovery findings that the top metabolic pathways affected by loss of MBLAC1 include primary bile acid biosynthesis and linoleate metabolism. Multiple other pathways were nominated as significantly impacted by MBLAC1 KO, though almost all of these derive from 2-3 molecules within their designated network. A notable exception is a pathway linked to urea cycle/amine group metabolism, where 9 of 38 features were nominated, though this pathway had not been identified in the earlier discovery analysis. In the validation analysis, a significant perturbation of GSH metabolism following loss of MBLAC1 was not identified. As the bile acid synthesis pathway, which retained significance, shares molecules with that of the GSH metabolic pathway, the lack of significance of the latter network may reflect an overall weaker effect of MBLAC1 genotype that becomes insignificant in the context of the more stringent, littermate based design of the validation experiment. Alternatively, this difference could derive from unknown variables associated with animal housing and husbandry at the two sites where samples were derived.

Potential Significance of Perturbation of Taurine-Derived Metabolites within the Primary Bile Acid Biosynthesis Pathway

As noted above, MBLAC1 KO appears to result in a consistent reduction in the abundance of many taurine derived metabolites such as taurochenodeoxycholic acid and taurocholate that reside in the primary bile acid metabolism pathway. Indeed, these features represent the most significantly altered and putatively identified metabolites in the dataset, with the greatest magnitude of change due to loss of MBLAC1. This pathway validation data provided additional support for bile acid biosynthesis and taurine derived metabolites as highly sensitive to MBLAC1 expression. Taurine and related metabolites have many important biological roles, ranging from essential contributions to bile acid conjugation in the liver, to the regulation of cardiac and skeletal muscle function, and evidence suggests that they can cross the blood brain barrier and regulate neurotransmission. Taurine has been shown to be protective against oxidative stress induced cell death in peripheral tissues such as liver in several animal models of hepatotoxicity. Likewise, tauroursodeoxycholic acid (TUDCA), a bile acid derivative of taurine, has been shown to be neuroprotective in in vitro and in vivo models of cell death such as retinal degeneration where the compound has been found to markedly decrease retinal neural cell death by reducing cellular stress and preventing release of pro-apoptotic factors. Therefore, loss of these molecules from the serum of MBLAC1 KO mice may indicate a role played by the MBLAC1 substrate in triggering the induction of taurine metabolic pathways that protect against cell stress and cell death. Chronic Cef treatment of cells has previously been reported to act via a Nrf2 pathway to induce expression of the cysteine/Glu exchanger and the Na+-dependent Glu transporters that can diminish the threat of excitotoxic insults and oxidative stress. It is hypothesized that short term Cef blockade of MBLAC1 is detected as a stressful event by Nrf2, whereas the lifelong absence of MBLAC1 may preclude cells from mounting an appropriate stress response, as revealed in a reduction in bile acid pathway molecules in the serum of MBLAC1 KO mice.

Potential Significance of Alterations in Linoleate Metabolism

In the validation analysis, it was confirmed that linoleate metabolism is one of the metabolic pathways sensitive to loss of MBLAC1. Linoleic acid is an essential poly-unsaturated, omega-6 fatty acid (PUFA) primarily known as a precursor for the biosynthesis of arachidonic acid. Alterations in linoleic acid levels have been associated with a wide variety of health consequences ranging from perturbations of skin and hair health, as well as obesity and cardiovascular disease. As changes in the metabolites of the linoleic acid metabolism pathway in MBLAC1 KO mice were observed, it is hypothesized that MBLAC1 KO mice may be more susceptible to abnormal brain health, a hypothesis that can be assessed through disease-mimicking pharmacological and genetic challenges.

### SUMMARY

Using an unbiased metabolomic approach, based on an UPLC-MS/MS, serum metabolome changes arising from constitutive elimination of MBLAC1, an enzyme of as yet undetermined function, were evaluated. Ninety-two annotations were assigned to features of interest that significantly differed in abundance in the serum of MBLAC1 KO mice compared to WT controls. MetaboAnalyst 3.0 and KEGG pathway analysis nominated multiple metabolic pathways impacted in the KO, with several linked to neuroprotective, oxidative stress reducing pathways. In an independent validation study, an impact of loss of MBLAC1 on bile acid biosynthesis and linoleate metabolism, pathways that share cell protective actions in the face of metabolic and oxidative cellular stress, was confirmed. These studies designate metabolic pathways that should be pursued in future, targeted analyses and that may ultimately reveal the endogenous substrate(s) for MBLAC1/SWIP-10. These networks were identified in serum, however, other networks can be identified through similar methods in, for example, urine, brain fluid and brain tissue. The reported neuroprotective actions of Cef, a demonstrated MBLAC1 ligand, may derive from the induction of cell defense mechanisms such as those designed to limit oxidative stress, effects that cannot be sustained in the context of a full loss of the enzyme.

### Example 3—Effect of Repeated Cef on Cocaine-Induced Locomotion

Methods:

Animals: All mice were used in accordance with protocols approved by the Florida Atlantic University Institutional Animal Care and Use Committee. Mice were group-housed on a 12 h/12 h light/dark cycle and given ad libitum access to food and water. Breeding was accomplished with mating of heterozygous males with heterozygous females. Male and female mice 11-13 weeks old at the start of the experiment were used in testing the effects of repeated ceftriaxone on response to cocaine in the open field assay. Data from males and females were combined.

Repeated ceftriaxone treatment/acute cocaine administration: MBLAC1 wild-type (WT, MBLAC1^{+/+}) and knockout mice (MBLAC1^{-/-}) were used in this experiment. Mice were injected IP once daily for 10 days with 200 mg/kg ceftriaxone (CEF) or saline. Twenty-four hours following the last CEF injection, mice were injected with 10 mg/kg cocaine or saline followed immediately by testing in open field activity chambers (Med Associates, Fairfax, Vt.) for 60 minutes. Locomotor activity was evaluated by photocells in the X, Y and Z dimensions. Horizontal beam breaks, vertical beam breaks, and repetitive movements were recorded and measures, including distance traveled, vertical rearing and stereotypic counts, were calculated. Data were analyzed in 5 min time bins within session, and for the first 30 min following cocaine injection. Data were analyzed by two-way ANOVA repeated measures, with time bin as the repeated measure, or by two-way ANOVA for 30 min cumulated data. At the end of behavior, mice were euthanized by CO₂.

The methods were performed essentially as described in Tallarida et al. Neurosci Lett vol. 556, p. 155-159, 2013.

Results:

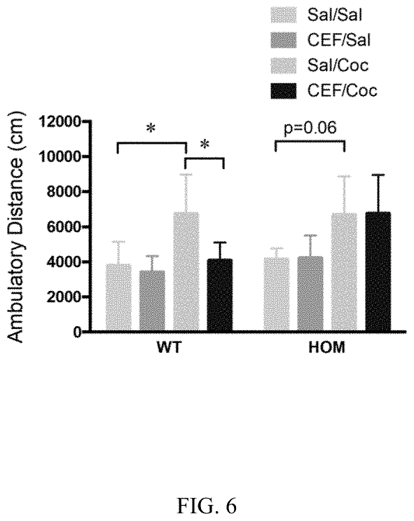

MBLAC1 WT and MBLAC1 KO mice responded to cocaine with an increase in total ambulatory distance in the open field in the first 30 min of a 60 min session compared to mice injected with saline (FIG. 6). Ten days of pretreatment with CEF completely blocked the effects of cocaine on MBLAC1 WT mice. However, the repeated CEF treatment had no effect on the response to cocaine in MBLAC1 KO mice.

### Example 4—Cocaine Sensitization

Methods:

Animals: All mice were used in accordance with protocols approved by the Florida Atlantic University Institutional Animal Care and Use Committee. Mice were group-housed on a 12 h/12 h light/dark cycle and given ad libitum access to food and water. Breeding was accomplished with mating of heterozygous males with heterozygous females. Male and female mice 11-13 weeks old at the start of the experiment were used in testing the effects of repeated cocaine in the open field assay. Data from males and females were combined.

Repeated cocaine administration: Mblac1 wild-type (WT, Mblac1^{+/+}) and knockout (KO) mice (HOM, Mblac1^{-/-}) were used in this experiment. On day 1, mice were placed in the open field activity chambers (Med Associates, Fairfax, Vt.) for 30 min as a habituation. On day 2, no behavior was performed. On day 3, mice were placed in the open field for 30 min, removed and administered a saline injection, and placed back in the open field for 60 min. On day 4, no behavior was performed. On day 5-9, mice were placed in the open field for 30 min, removed and administered 10 mg/kg cocaine, IP, and placed back in the open field for 60 min. Then, on day 10, mice were placed in the open field for 30 min, removed and administered a saline injection, and placed back in the open field for 60 min. This was followed by a two-week abstinence period during which mice receive no treatments. This was followed by a single day on which mice are placed in the open field for 30 min, removed and administered a cocaine injection, and placed back in the open field for 60 min. At the end of behavior, mice were euthanized by CO₂. Locomotor activity was evaluated by photocells in the X, Y and Z dimensions. Horizontal beam breaks, vertical beam breaks, and repetitive movements were recorded and measures, including distance traveled, vertical rearing and stereotypic counts, were calculated. Data were analyzed in 5 min time bins within session, and for the 60 min following cocaine injection. Data were analyzed by two-way ANOVA repeated measures, with time bin as the repeated measure, or by two-way ANOVA for 60 min cumulative data. At the end of behavior, mice were euthanized by CO₂.

Results:

On the first day of cocaine administration, MBLAC1 HOM mice responded to cocaine with an increase in total ambulatory distance in the open field compared to mice injected with saline (FIG. 7A). The effect of cocaine on activity in MBLAC1 WT mice was not significant, suggesting that HOM are more sensitive to cocaine than WT mice (FIG. 7A). Two weeks later, a single injection of cocaine resulted in increased open field activity in both WT and HOM mice; however, the increase in activity was greater in HOM compared to WT mice (FIG. 7B). Furthermore, the cocaine-induced open field activity after two weeks was greater in HOM compared to the effect on day of cocaine for this group, whereas this was not different for WT mice. Thus, HOM mice demonstrate a sensitized effect of cocaine at two weeks that was not observed in WT. Taken together, these data support an enhanced response of HOM mice to the effects of cocaine, and an increased development of sensitization.

### Other Embodiments

Any improvement may be made in part or all of the method steps. All references, including publications, patent applications, and patents, cited herein are hereby incorporated by reference. The use of any and all examples, or exemplary language (e.g., “such as”) provided herein, is intended to illuminate the invention and does not pose a limitation on the scope of the invention unless otherwise claimed. Any statement herein as to the nature or benefits of the invention or of the preferred embodiments is not intended to be limiting, and the appended claims should not be deemed to be limited by such statements. More generally, no language in the specification should be construed as indicating any non-claimed element as being essential to the practice of the invention. This invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contraindicated by context.

What is claimed is:

1. A method of identifying molecular networks in an animal that are modulated by Cef in the presence of MBLAC1 but not in the absence of MBLAC1, the method comprising the steps of:
(a) providing a group of test MBLAC1 knock-out (KO) animals, a group of control MBLAC-1 KO animals, a group of test wild-type (WT) MBLAC1 animals and a group of control WT MBLAC1 animals, or cells from each group of animals;
(b) administering a dose of Cef to the group of test MBLAC1 KO animals and to the group of test WT MBLAC1 animals, or to cells from the group of test MBLAC1 KO animals and to cells from the group of test WT MBLAC1 animals at least once under conditions known to reduce effects of a substance of abuse in an animal;
(c) collecting biological samples from all the groups of animals and analyzing levels of nucleic acids, neurotransmitters, proteins, and metabolites in the biological samples or analyzing levels of nucleic acids, neurotransmitters, proteins, and metabolites in the cells from all the groups of animals, resulting in a plurality of test MBLAC1 KO molecular networks, a plurality of control MBLAC1 KO molecular networks, a plurality of test WT MBLAC1 molecular networks, and a plurality of control WT MBLAC1 molecular networks;
(d) comparing the plurality of test WT MBLAC1 molecular networks to the plurality of control WT MBLAC1 molecular networks and identifying any molecular networks present in the test WT MBLAC1 animals but not in the control WT MBLAC1 animals as Cef-responsive molecular networks;
(e) comparing the Cef-responsive molecular networks to the plurality of test MBLAC1 KO molecular networks and to the plurality of control MBLAC1 KO molecular networks and identifying any Cef-responsive molecular networks that are not present in the plurality of test MBLAC1 KO molecular networks or in the plurality of control MBLAC1 KO molecular networks, or that are overrepresented relative to the plurality of test MBLAC1 KO molecular networks, as molecular networks that are modulated by Cef in the presence of MBLAC1 but not in the absence of MBLAC1.

2. The method of claim 1, wherein the molecular networks that are modulated by Cef in the presence of MBLAC1 but not in the absence of MBLAC1 comprise a statistically significant number of nucleic acids, neurotransmitters, proteins, and/or metabolites whose levels are modulated by Cef in the presence of MBLAC1 relative to nucleic acids, neurotransmitters, proteins, and/or metabolites whose levels are not modulated by Cef in the absence of MBLAC1.

3. The method of claim 1, wherein steps (c)-(d) comprise using software.

4. The method of claim 1, wherein the animals are rodents.

5. The method of claim 1, wherein the molecular networks that are modulated by Cef in the presence of MBLAC1 but not in the absence of MBLAC1 are associated with Cef's ability to act through MBLAC1 to reduce actions of at least one substance of abuse.

6. The method of claim 5, wherein the molecular networks that are modulated by Cef in the presence of MBLAC1 but not in the absence of MBLAC1 are relevant to at least one of: substance abuse and addiction.

7. The method of claim 1, wherein the biological samples are selected from the group consisting of: serum, cerebrospinal fluid and brain tissue.

8. The method of claim 1, wherein analyzing levels of nucleic acids, neurotransmitters, proteins, and metabolites in step (c) comprises at least one of: RNA sequencing, microarray analysis, epigenome analysis, proteomic analysis and metabolomics analysis.

9. A method of analyzing MBLAC1-dependent levels of molecules comprising analyzing levels of molecules in WT MBLAC1 animals or cells therefrom and levels of molecules in MBLAC1 KO animals or cells therefrom, comparing the levels of molecules in WT MBLAC1 animals or cells therefrom to the levels of molecules in MBLAC1 KO animals or cells therefrom and identifying any molecules whose levels of expression statistically significantly differ in the WT MBLAC1 animals or cells therefrom relative to the MBLAC1 KO animals or cells therefrom.

10. The method of claim 9, wherein the molecules are selected from at least one of: nucleic acids, neurotransmitters, proteins, and metabolites.

11. The method of claim 10, wherein analyzing the levels of the molecules comprises at least one of: RNA sequencing, microarray analysis, epigenome analysis, proteomic analysis and metabolomics analysis.

12. The method of claim 9, wherein the animals are rodents.

13. The method of claim 9, wherein the identified molecules whose levels of expression statistically significantly differ in the WT MBLAC1 animals or cells therefrom relative to the MBLAC1 KO animals or cells therefrom form a molecular network.

14. A method of identifying molecules and/or molecular networks thereof modulated by a substance of abuse and Cef in an MBLAC1 dependent manner, the method comprising the steps of:
(a) providing a group of control WT MBLAC1 animals, a first test group of WT MBLAC1 animals, a second test group of WT MBLAC1 animals, a third test group of WT MBLAC1 animals, a group of control MBLAC1 KO animals, a first group of test MBLAC1 KO animals, a second test group of MBLAC1 KO animals and a third test group of MBLAC1 KO animals;
(b) administering a substance of abuse to the first test group of WT MBLAC1 animals and collecting biological samples from the group of control WT MBLAC1 animals and from the first test group of WT MBLAC1 animals;
(c) analyzing levels of molecules selected from the group consisting of: nucleic acids, neurotransmitters, proteins, and metabolites in the biological samples and determining differences in the levels of the molecules between the group of control WT MBLAC1 animals and the first test group of WT MBLAC1 animals;
(d) administering a dose of Cef to the second test group of WT MBLAC1 animals and administering the same dose of Cef and the substance of abuse to the third test group of WT MBLAC1 animals, wherein the dose of Cef is administered at least once under conditions known to reduce effects of the substance of abuse in an animal;
(e) collecting biological samples from the second test group of WT MBLAC1 animals and the third test group of WT MBLAC1 animals after administration of the dose of Cef and the substance of abuse;
(f) analyzing levels of molecules selected from the group consisting of: nucleic acids, neurotransmitters, proteins, and metabolites in the biological samples of step (e) and determining differences in the levels of the molecules between the second test group of WT MBLAC1 animals and the third test group of WT MBLAC1 animals;
(g) comparing the differences in the levels of step (c) to the differences in the levels of step (f) to identify any molecules or one or more molecular networks thereof whose levels are modulated by Cef and by the substance of abuse;
(h) administering a substance of abuse to the first test group of MBLAC1 KO animals and collecting biological samples from the group of control MBLAC1 KO animals and from the first test group of MBLAC1 KO animals;
(i) analyzing levels of molecules selected from the group consisting of: nucleic acids, neurotransmitters, proteins, and metabolites in the biological samples of step (h) and determining differences in the levels of the molecules between the group of control MBLAC1 KO animals and the first test group of MBLAC1 KO animals;
(j) administering a dose of Cef to the second test group of MBLAC1 KO animals and administering the same dose of Cef and the substance of abuse to the third test group of MBLAC1 KO animals, wherein the dose of Cef is administered at least once under conditions known to reduce effects of the substance of abuse in an animal;
(k) collecting biological samples from the second test group of MBLAC1 KO animals and the third test group of MBLAC1 KO animals after administration of the dose of Cef and the substance of abuse;
(l) analyzing levels of molecules selected from the group consisting of: nucleic acids, neurotransmitters, proteins, and metabolites in the biological samples of step (k) and determining differences in the levels of the molecules between the second test group of MBLAC1 KO animals and the third test group of MBLAC1 KO animals;
(m) comparing the differences in the levels of step (i) to the differences in the levels of step (l) to identify any molecules or one or more molecular networks thereof that modulate actions of the substance of abuse in any MBLAC1 KO animal that has been administered the substance of abuse; and
(n) comparing the molecules or one or more molecular networks thereof whose levels are modulated by Cef and by the substance of abuse of step (g) to the molecules or one or more molecular networks thereof that modulate actions of the substance of abuse in any MBLAC1 KO animal of step (m) and identifying molecules or one or more molecular networks thereof whose levels are modulated by Cef and by the substance of abuse in an MBLAC1-dependent manner.

15. The method of claim 14, wherein the substance of abuse is selected from the group consisting of: cocaine, amphetamine, morphine, ethanol, methamphetamine, clorazepate, cathinones, bath salts, heroin, nicotine, alcohol, ketamine, and MDMA.

16. The method of claim 14, wherein the molecules or one or more molecular networks thereof whose levels are modulated by Cef and by the substance of abuse in an MBLAC1-dependent manner are relevant to at least one of: substance abuse and addiction.

17. The method of claim 14, wherein steps (c), (f), (g), (i), (l), (m) and (n) are performed using network software.

18. The method of claim 14, wherein the animals are rodents.

19. The method of claim 14, further comprising the step of identifying at least one drug target from the molecules or one or more molecular networks thereof whose levels are modulated by Cef and by the substance of abuse in an MBLAC1-dependent manner.

20. The method of claim 19, further comprising testing the at least one drug target for an ability to attenuate or block the actions of the substance of abuse.

21. A method for identifying a potential therapeutic drug target for substance abuse treatment comprising: identifying at least one nucleic acid, neurotransmitter, protein, or metabolite that is modulated by a substance of abuse and Cef in an MBLAC1-dependent manner as identified in the method of claim 7 as a potential drug target for treating abuse of the substance in a mammal.

22. The method of claim 21, further comprising screening candidate therapeutic drugs against the potential drug target.

23. An MBLAC1 KO animal lacking any functional copies of the Mblac1 gene.

24. The MBLAC1 KO animal of claim 23, wherein N-terminal coding sequences of the Mblac1 gene are disrupted by CRISPR/Cas9 resulting in a complete loss of MBLAC1 protein expression in the MBLAC1 knock-out animal.

25. The MBLAC1 KO animal of claim 23, wherein mRNA transcription or mRNA translation of the Mblac1 gene has been significantly reduced or eliminated by chemical or genetic means.

26. The MBLAC1 KO animal of claim 23, wherein the animal is a rodent.
